SEQUENCE LISTING (12 sequences)

SEQ ID NO: 1 - protein, 140 aa, Zea Mays
MAATALSMSILRAPPPCFSSPLRLRVAVAKPLAAPMRRQLLRAQATYNVKLITPEGEVELQVPDDVYILDFAEEEGIDLPFSCRAGSCSSCAGKVVSGSVDQSDQSFLNDNQVADGWVLTCAAYPTSDVVIETHKEDDLL

SEQ ID NO: 2 - protein, 150 aa, Zea Mays
MATVLGSPRAPAFFFSSSSLRAAPAPTAVALPAAKVGIMGRSASSRRRLRAQATYNVKLITPEGEVELQVPDDVYILDQAEEDGIDLPYSCRAGSCSSCAGKVVSGSVDQSDQSYLDDGQIADGWVLTCHAYPTSDVVIETHKEEELTGA

SEQ ID NO: 3 - protein, 150 aa, Zea Mays
MATVLGSPRAPAFFFSPSSLRAAPAPTAVALPAAKVGIMGRSASSRGRLRAQATYNVKLITPEGEVELQVPDDVYILDQAEEDGIDLPYSCRAGSCSSCAGKVVSGSVDQSDQSYLDDGQIAAGWVLTCHAYPTSDVVIETHKEEELTGA

SEQ ID NO: 4 - DNA, 423 bp, Zea Mays
atggccgccaccgccctgagcatgagcatcctccgcgcgccgccgccctgcttctcgtccccactcaggctcagggtcgcggttgccaagccgctggcggcccccatgcggcgccagctgctgcgcgcgcaggccacctacaacgtgaagctgatcacgccggagggggaggtggagctgcaggtgcccgacgacgtctacatactggacttcgccgaggaggaaggcatcgacctgcccttctcctgccgcgcggggtcctgctcctcctgcgccggcaaggtcgtctccggctccgtcgaccagtccgaccagagcttcctcaacgacaaccaggtcgccgacggctgggtgctcacctgcgctgcgtaccccacctccgacgtcgtcatcgagacgcacaaggaggatgacctcctataa

SEQ ID NO: 5 - DNA, 791 bp, Zea Mays
gtgtggccgcccgtgtcgtgtagtgtgtagtcgcagcagctagcgcccggccggccagtcgagtgagtccatcctccatcgccatccaatggccgccaccgccctgagcatgagcatcctccgcgcgccgccgccctgcttctcgtccccactcaggctcagggtcgcggttgccaagccgctggcggcccccatgcggcgccagctgctgcgcgcgcaggccacctacaacgtgaagctgatcacgccggagggggaggtggagctgcaggtgcccgacgacgtctacatcctggacttcgccgaggaggaaggcatcgacctgcccttctcctgccgtgcggggtcctgctcctcctgcgccggcaaggtcgtctctggctccgtcgaccagtccgaccagagcttcctcaacgacaaccaggtcgccgacggttgggtgctcacctgcgctgcgtaccccacctccgacgtcgtcatcgagacgcacaaggaggatgacctcctataattctagctagctatacaccgccagggcccgtcgtcttgtgccaccacatgcagtaccgcccgcgcaggagatgagacgtgtcgtctcaataattctagctatatatatatatatgcatgcatgcatgtacttttccctgttccaaactgagtatattctaaattacaagatttaatcacaaggtttagagcaactccaaccatgagtctcataattggctctatattttgatttagcaactcacttaatttgtttaagatctaaacacatgttttgtttc

SEQ ID NO: 6 - DNA, 453 bp, Zea Mays
atggccaccgtcctgggcagcccccgcgcgccggccttcttcttctcgtcgtcctccctccgcgccgcgccggcgcctaccgccgtggcgctgcctgcggccaaggtgggcatcatgggccgtagcgccagcagcaggcgcaggctgcgcgcgcaggccacctacaacgtgaagctgatcacgccagagggggaggtggagctgcaggtgcccgacgacgtgtacatcctggaccaggccgaggaggacggcatcgacctgccctactcctgccgcgcggggtcctgctcctcgtgcgccggcaaggtcgtctccggctccgtggaccagtccgaccagagctacctcgacgacggccagatcgccgacggctgggtgctcacctgccacgcctaccccacctctgacgtcgtcatcgagacgcacaaggaggaggagctcaccggcgcataa

SEQ ID NO: 7 - DNA, 453 bp, Zea Mays
atggccaccgtcctaggcagcccccgcgcgccggccttcttcttctcgccgtcctccctccgtgccgcgccggcgcccaccgccgtggcgctgcctgcggccaaggtgggcatcatgggccgtagcgccagcagcaggggcaggctgcgcgcgcaggccacctacaacgtgaagctgatcacgccggagggggaggtggagctgcaggtgcccgacgacgtgtacatcctggaccaggccgaggaggacggcatcgacctgccctactcctgccgcgcggggtcctgctcctcctgcgccggcaaggtcgtctccggctccgtggaccagtccgaccagagctacctcgacgacggccagatcgccgccggctgggtgctcacctgccacgcctaccccacctctgacgtcgtcatcgagacgcacaaggaggaggagctcaccggcgcataa

SEQ ID NO: 8 - protein, 144 aa, Sorghum bicolor
MATALSSLRAPAAFSLGIAAAPAPAAAATVVALPAAKPARGARLRAQATYNVKLITPDGEVELQVPDDVYILDQAEEEGIDLPFSCRAGSCSSCAGKVVSGTVDQSDQSFLDDAQVEGGWVLTCAAYPTSDVVIETHKEEDLVG

SEQ ID NO: 9 - protein, 151 aa, Saccharum hybrid
MSTSTFATSCTLLGNVRTQASQAAVKSPSSLSFFSQVMKVPSLKTSKKLDVSAMAVYKVKLVTPEGQEHEFDAPDDTYILDAAETAGVELPYSCRAGACSTCAGKIESGAVDQSDGSFLDDGQQEEGYVLTCVSYPKSDCAIHTHKEGDLY

SEQ ID NO: 10 - protein, 151 aa, Panicum hallii
MSISTFATSCVLLSNVRTQTSQTPVKSPSSLSFFSQGMKVPSLKTSKKLDVSAMAVYKVKLVTPEGVEHEFEAPDDTYILDAAETAGVELPYSCRAGACSTCAGKIEAGEVDQSDGSFLDDGQQAEGYVLTCVSYPKSDCVIHTHKEGDLY

SEQ ID NO: 11 - protein, 148 aa, Arabidopsis thaliana
MASTALSSAIVGTSFIRRSPAPISLRSLPSANTQSLFGLKSGTARGGRVTAMATYKVKFITPEGELEVECDDDVYVLDAAEEAGIDLPYSCRAGSCSSCAGKVVSGSVDQSDQSFLDDEQIGEGFVLTCAAYPTSDVTIETHKEEDIV

SEQ ID NO: 12 - DNA, 985 bp, Arabidopsis thaliana
gtgtgagctgtcccaagtaagaccacgtaatactcacctcaacaagatagtgttcttaaagtgtgtcaaacacaatcacacacacacaaatcataaaacacaaagacgataatccatcgatccacagaatagacgccacgtggtagataggattctcactaaaaagttctcaccttttaatctttctccacgccatttccacaagccataatcctcaaaaatctcaactttatctcccaaaacacaaaacaaaaaaaaatggcttccactgctctctcaagcgccatcgtcggaacttcattcatccgtcgttccccagctccaatcagtctccgttcccttccatcagccaacacacaatccctcttcggtctcaaatcaggcaccgctcgtggtggacgtgtcacagccatggctacatacaaggtcaagttcatcacaccagaaggtgagctagaggttgagtgtgacgacgacgtctacgttcttgatgctgctgaggaagctggaatcgatttgccttactcttgccgtgctggttcttgttcgagctgtgctggtaaagttgtgtctggatctgttgatcagtctgaccagagtttccttgatgatgaacagattggtgaagggtttgttctcacttgtgctgcttaccctacctctgatgttaccattgaaacccacaaagaagaagacattgtttaagcctcacctactcaccagcttttgatggtttaaaaatcatgtctttataaatttcacattttgggttgagtttgttgttactaaaaactattgttatctgttgttattgttcctggtttggctcaccatcaatcgatgacattttaaactatgcaactgcaaattctgcaacactttcgatgagaatctaacattatcgtttaaacattggaaatacattttcttgaagtctagctagctttggtttgtagttcttattctgaactcaacaatcatcaaa

(19) United States
(12) Patent Application Publication
(10) Pub. No.: US 2021/0024948 A1
(43) Pub. Date: Jan. 28, 2021

(54) ENHANCEMENT OF PHOTOSYNTHETIC RATES, ABIOTIC STRESS TOLERANCE AND BIOMASS YIELD THROUGH EXPRESSIOPN OF A C4 PLANT FERREDOXIN IN C3 PHOTOSYNTHETIC PLANTS

(71) Applicant: New Mexico Consortium, Los Alamos, NM (US)

(72) Inventors: Richard T. Sayre, Los Alamos, NM (US); Panagiotis Lymperopoulos, Los Alamos, NM (US); Choon-Hwan Lee, Los Alamos, NM (US); Guangxi Wu, Los Alamos, NM (US)

(21) Appl. No.: 17/042,803

(22) PCT Filed: Mar. 28, 2019
(86) PCT No.: PCT/US2019/024669

§ 371 Date: Sep. 28, 2020

Related U.S. Application Data
(60) Provisional application No. 62/649,239, filed on Mar. 28, 2018.

(51) Int. Cl. C12N 15/82

(52) U.S. Cl. CPC C12N 15/8269

(57) ABSTRACT

The invention include systems, methods, and compositions related to the enhancement of photosynthetic electron transfer rates, abiotic stress tolerance, CO₂ fixation rates, and increases in yield/biomass in plants. These methods and associated transgenic plants encompass the expression, or overexpression, of one or more genes that improve photosynthetic electron transfer rates, abiotic stress tolerance, CO₂ fixation rates, and yield/biomass in plants. Such enhanced plant characteristics may be achieved through the expression, or overexpression of select photosynthetic ferredoxin proteins in a plant or plant cell. In certain embodiments, such enhanced plant characteristics may be achieved through the expression, or overexpression, of one or more photosynthetic ferredoxin proteins from a C4 plant in a C3 plant or plant cell.

This application claims the benefit of and priority to U.S. Provisional Application No. 62/649,239, filed Mar. 28, 2018, which is incorporated herein by reference in its entirety.

### SEQUENCE LISTING

The instant application contains a Sequence Listing which has been submitted electronically in ASCII format and is hereby incorporated by reference in its entirety.

### TECHNICAL FIELD

The field of the present invention relates generally to plant molecular biology and plant biotechnology. More specifically, the invention relates to systems, methods, and compositions to generate genetically modified plants having enhanced physiological characteristics. In particular, the invention relates to genetically modified plants having enhanced photosynthetic electron transfer rates, abiotic stress tolerance, CO₂ fixation rates, and enhanced biomass.

### BACKGROUND

Plants are generally classified into C3 plants, C4 plants, based on the kind of initial fixed products during photosynthetic fixation of CO₂. Ninety percent or more of plants on the earth belong to C3 plants, which include, for example, agriculturally important plants such as rice and barley. The photosynthetic pathway of C3 plants is also called the Calvin pathway, and an enzyme involved in photosynthetic fixation of CO₂ in this pathway is ribulose-1,5-bisphosphate carboxylase (RuBisCO). This enzyme has an affinity for both CO₂ and for O₂. Therefore, CO₂ is subjective to competitive inhibition by oxygen in the C3 photosynthetic pathway. The C4 plants are those which have evolved to overcome such non-efficient photosynthetic fixation of oxygen The C4 plants have a mechanism for concentrating CO₂ thus competitively inhibiting the oxygenase reaction of RuBisCO. An enzyme involved in photosynthetic fixation of CO₂ in the photosynthetic pathway of the C4 plants is phosphoenolpyruvate carboxylase (PEPC). This enzyme has a high capacity of photosynthetic fixation of CO₂ without its activity being inhibited by O₂. The product of CO₂ fixation by PEPC is oxaloacetic acid which is then either reduced to malate transaminated to produce aspartic acid. These reactions occur specifically in the mesophyll cells of the leaf. The C4 acids (malate and/or aspartate) are then transferred to the inner bundle sheath cells (BSC) where they are decarboxylated releasing CO₂ and elevating the internal CO₂ concentration in the BSC to approximately 10× that of the atmosphere. RuBisCO is expressed only in the BSC chloroplasts. Thus, the elevated CO₂ concentration in these cells competitively inhibits the oxygenase reaction improving photosynthetic efficiency. Algae also elevate the internal CO₂ concentration in chloroplasts by actively transporting bicarbonate into the cells using ATP where it is subsequently dehydrated to produce CO₂ in the chloroplast, competitively inhibiting the oxygenase reaction and enhancing photosynthetic efficiency. It is expected that the photosynthetic capacity and productivity of the agriculturally important C3 plants (e.g., rice) will be remarkably improved by providing a C3 plant with the photosynthetic function of a C4 plant or the more simple bicarbonate pumping system of algae. As shown below, one aspect of the current invention includes the expression of algal CO₂ concentrating systems in C3 plants. In addition, it was proposed that the expression of specific Ferredoxins that would enhance cyclic photophosphorylation with the original intention to increase ATP synthesis to support the ATP requirement of the algal plasma membrane ATP-dependent bicarbonate transporter, HLA, are shown to be sufficient in the absence of the expression of other genes of the algal bicarbonate transporter system to support elevated CO₂ fixation rates alone by substantially elevating rates of linear electron transfer to support CO₂ fixation.

Ferredoxins (Fds) are small soluble electron carrier proteins. In the final reaction of photosynthetic electron transfer (PET), photosystem I (PSI) donates electrons to Fd, which acts as the soluble electron donor to various acceptors in the chloroplast stroma and can also return electrons to the thylakoid in cyclic electron flow (CET). The electron cascade to supply carbon fixation requires photoreduction of NADP by Fd, catalyzed by Fd-NADP(H) oxidoreductase (FNR). Many other plastid enzymes accept electrons directly from Fd for metabolic processes. These include, but are not limited to, nitrite reductase and sulfite reductase, which are essential for assimilation of inorganic nitrogen and sulfur, respectively; and Fd-dependent glutamine oxoglutarate aminotransferase and fatty acid desaturase, which catalyze key steps in amino acid and fatty acid metabolism, respectively. In addition, Fd donation to thioredoxin via the Fd:thioredoxin reductase translates the redox state of the electron transfer chain into a regulatory signal controlling the activity of many enzymes. Fds are also capable of accepting electrons from NADPH via FNR, in a reversal of the photosynthetic reaction, allowing electron donation from reduced Fd to different acceptors under non-photosynthetic conditions.

Generally, higher plants possess genes for several different Fd isoproteins. In the C4-plant maize (Zea mays), for example, different functions have been identified for at least two of the leaf-type Fds, namely ferredoxin-1 (Fd1) and ferredoxin-1 (Fd2). These two ferredoxin isoproteins are generally restricted to leaves, and their accumulation may be induced by light. Thus, they are referred to as photosynthetic ferredoxins. As generally outlined in FIG. 10, there is a higher demand for ATP (which is disproportionately produced by cyclic electron transfer, CET) in the bundle sheath cells of NADP malic enzyme type C4 plants, and maize Fd1 and Fd2 are differentially expressed in mesophyll and bundle sheath cells, respectively. Fd2 has decreased affinity for FNR and demonstrates a higher activity in CET around the photosystems, whereas Fd1 predominantly drives linear electron flow. (See Voss I, Goss T, Murozuka E, et al., FdC1, a Novel Ferredoxin Protein Capable of Alternative Electron Partitioning, Increases in Conditions of Acceptor Limitation at Photosystem I. The Journal of Biological Chemistry. 2011; 286(1):50-59.) In C3 plants, this spatial distribution is not observed, but duplicate photosynthetic Fds are still present, and there is some evidence that these proteins also act differentially in linear electron flow and CET. Despite the fact that Fd1 and Fd2 have a similar affinity for FNR, they appear to perform different functions in photosynthesis, and there is evidence that Fd1 makes a specifically higher contribution to CET.

Being central to photosynthesis, as well as other critical biosynthetic and energy pathways in plants, there is a need to improve plant productivity through modifications in these Fd-dependent pathways. As explained below, it is expected that the ability to enhance photosynthetic carbon assimilation in C3 plants through the incorporation of C4 Fd-photosynthetic components may result in enhanced plant yields and biomass, improved photosynthesis efficiency, increased carbon fixation, and greater abiotic tolerance among other attributes.

### SUMMARY OF THE INVENTION(S)

The invention include systems, methods, and compositions related to the enhancement of photosynthetic electron transfer rates, abiotic stress tolerance, CO₂ fixation rates, and increases in yield/biomass in plants. These methods and associated transgenic plants encompass the expression, or overexpression, of one or more genes that improve photosynthetic electron transfer rates, abiotic stress tolerance, CO₂ fixation rates, and yield/biomass in plants. Such enhanced plant characteristics may be achieved through the expression, or overexpression of select photosynthetic Fd proteins in a plant or plant cell. In certain embodiments, such enhanced plant characteristics may be achieved through the expression, or overexpression, of one or more photosynthetic Fd proteins from a C4 plant in a C3 plant or plant cell.

Compositions and methods for increasing plant growth, enhancing photosynthesis, increasing abiotic stress resistance and increasing crop yield and biomass are provided. The methods involve the heterologous expression in a C3 plant or cell of interest of at least one C4 photosynthetic Fd sequence—such term generally referring to a polynucleotide encoding a photosynthetic Fd, or an amino acid sequence of a photosynthetic Fd. C3 plants showing heterologous expression of one or more C4 photosynthetic Fd coding sequence of interest are encompassed by the invention. It is recognized that any method for the heterologous expression of a C4 photosynthetic Fd coding sequences in a plant of interest can be used in the practice of the methods disclosed herein. Such methods include transformation, breeding and the like. Heterologous expression of the C4 photosynthetic Fd coding sequences in the plant of interest results in the enhanced characteristics disclosed generally herein. Expression cassettes and vectors comprising the C4 Fd sequences disclosed herein are also provided herein as generally described below.

One aim of the invention may include a genetically modified C3 plant expressing a heterologous photosynthetic Fd, or a variant thereof, from a C4 plant. Expression of a heterologous Fd polynucleotide from a C4 plant may confer to a C3 plant enhanced photosynthetic characteristics, such as enhanced photosynthetic electron transfer, and photosynthetic CO₂ fixation rates. Expression of a heterologous Fd polynucleotide from a C4 plant may confer to a C3 plant enhanced abiotic (light and/or heat) stress resistance. Expression of a heterologous Fd polynucleotide from a C4 plant may confer to a C3 plant enhanced yields. Expression of a heterologous Fd polynucleotide from a C4 plant may confer to a C3 plant enhanced biomass. Embodiments of the invention may include increased plant yield and biomass, which in some embodiments may be up to, and even greater than a 2-fold increase in above ground biomass yield in Fd transgenic C3 plants compared to wild type or control plants.

Another aim of the invention may include the expression of a heterologous Fd polynucleotide from a C4 plant in a C3 plant that may further specifically confer to a C3 plant enhanced tolerance to abiotic stress, such as low temperature and high light stress as measured by reduced damage to photosystem II, enhanced levels of non-photochemical quenching (NPQ), accelerated dark-dependent decay rates of NPQ, elevated levels of open photosystem II complexes, and increased linear or cyclic electron transfer rates following stress application compared to wild type or control plants.

One aim of the invention may include a genetically modified C3 plant expressing a heterologous photosynthetic Fd1, or a variant thereof from a C4 plant. Expression of a heterologous photosynthetic Fd1 polynucleotide from a C4 plant may confer to a C3 plant enhanced photosynthetic characteristics, such as enhanced photosynthetic electron transfer, and photosynthetic CO₂ fixation rates. Expression of a heterologous photosynthetic Fd1 polynucleotide from a C4 plant may confer to a C3 plant enhanced abiotic stress resistance. Expression of a heterologous photosynthetic Fd1 polynucleotide from a C4 plant may confer to a C3 plant enhanced yields. Expression of a heterologous photosynthetic Fd1 polynucleotide from a C4 plant may confer to a C3 plant enhanced biomass. Embodiments of the invention may include increased plant yield and biomass, which in some embodiments may be up to, and even greater than a 2-fold increase in above ground biomass yield in Fd1 transgenic plants compared to wild-type or control plants.

Another aim of the invention may include the expression of a heterologous Fd1 polynucleotide from a C4 plant in a C3 plant that may further specifically confer to a C3 plant enhanced tolerance to abiotic stress, such as low temperature and high light stress as measured by reduced damage to photosystem II, enhanced levels of non-photochemical quenching (NPQ), accelerated dark-dependent decay rates of NPQ, elevated levels of open photosystem II complexes, and increased cyclic electron transfer rates following stress application compared to wild type or control plants.

One aim of the invention may include a genetically modified C3 plant expressing a heterologous photosynthetic Fd2, or a variant thereof from a C4 plant. Expression of a heterologous Fd2 polynucleotide from a C4 plant may confer to a C3 plant enhanced photosynthetic characteristics, such as enhanced photosynthetic electron transfer, and photosynthetic CO₂ fixation rates. Expression of a heterologous Fd2 polynucleotide from a C4 plant may confer to a C3 plant enhanced abiotic stress resistance. Expression of a heterologous Fd2 polynucleotide from a C4 plant may confer to a C3 plant enhanced yields. Expression of a heterologous Fd2 polynucleotide from a C4 plant may confer to a C3 plant enhanced biomass. Embodiments of the invention may include increased plant yield and biomass, which in some embodiments may be up to, and even greater than a 2-fold increase in above ground biomass yield in Fd2 transgenic plants compared to wild type or control plants.

Another aim of the invention may include the expression of a heterologous Fd2 polynucleotide from a C4 plant in a C3 plant that may further specifically confer to a C3 plant enhanced tolerance to abiotic stress, such as low temperature and high light stress as measured by reduced damage to photosystem II, enhanced levels of non-photochemical quenching (NPQ), accelerated dark-dependent decay rates of NPQ, elevated levels of open photosystem II complexes, and increased linear electron transfer rates following stress application compared to wild type plants or control plants.

One aim of the invention may include a genetically modified C3 plant co-expressing a heterologous photosynthetic Fd2 and Fd1, or variants thereof from a C4 plant. Expression of a heterologous Fd2 and Fd1 polynucleotide(s) from a C4 plant may confer to a C3 plant enhanced photosynthetic characteristics, such as enhanced photosynthetic electron transfer, and photosynthetic CO₂ fixation rates. Co-expression of a heterologous Fd2 and Fd1 polynucleotide(s) from a C4 plant may confer to a C3 plant enhanced abiotic stress resistance. Co-expression of a heterologous Fd2 and Fd1 polynucleotide(s) from a C4 plant may confer to a C3 plant enhanced yields. Co-expression of a heterologous Fd2 and Fd1 polynucleotide(s) from a C4 plant may confer to a C3 plant enhanced biomass. Embodiments of the invention may include increased plant yield and biomass, which in some embodiments may be up to, and even greater than a 2-fold increase in above ground biomass yield in Fd2 and Fd1 transgenic plants compared to wild type plants or control plants.

Another aim of the invention may include the co-expression of a heterologous Fd2 and Fd1 polynucleotide(s) from a C4 plant in a C3 plant that may further specifically confer to a C3 plant enhanced tolerance to abiotic stress, such as low temperature and high light stress as measured by reduced damage to photosystem II, enhanced levels of non-photochemical quenching (NPQ), accelerated dark-dependent decay rates of NPQ, elevated levels of open photosystem II complexes, and increased linear and cyclic electron transfer rates following stress application compared to wild type plants or control plants.

Another aim of the invention may include the expression of a bundle sheath cell specific Fd2 in a C3 plant, such as an oil seed or oil crop. In this embodiment, a preferred oil crop may be Camelina sativa. Expression of this heterologous maize bundle sheath cell specific Fd2 gene may confer to a Camelina sativa plant enhanced tolerance to low temperature and high light stress as measured by reduced damage to photosystem II, enhanced levels of non-photochemical quenching (NPQ), accelerated dark-dependent decay rates of NPQ, elevated levels of open photosystem II complexes, and increased linear electron transfer rates following stress application compared to wild type plants. Embodiments of the invention may include increased biomass, which in some embodiments may be up to, and even greater than a 2-fold increase in above ground biomass yield in Fd2 transgenic plants compared to control plants.

Another aim of the invention may include the expression of a mesophyll cell specific Fd1 in a C3 plant, such as an oil seed or oil crop. In this embodiment, a preferred oil crop may be Camelina sativa. Expression of this heterologous maize bundle sheath cell specific Fd1 gene may confer to a Camelina sativa plant enhanced tolerance to low temperature and high light stress as measured by reduced damage to photosystem II, enhanced levels of non-photochemical quenching (NPQ), accelerated dark-dependent decay rates of NPQ, elevated levels of open photosystem II complexes, and increased cyclic electron transfer rates following stress application compared to wild type plants. Embodiments of the invention may include increased biomass, which in some embodiments may be up to, and even greater than a 2-fold increase in above ground biomass yield in Fd1 transgenic plants compared to control plants.

Another aim of the invention may include the expression of a maize (Zea mays) bundle sheath cell specific Fd2 in a C3 plant, such as an oil seed or oil crop. In this embodiment, a preferred oil crop may be Camelina sativa. Expression of this heterologous maize bundle sheath cell specific Fd2 gene may confer to a Camelina sativa plant enhanced tolerance to low temperature and high light stress as measured by reduced damage to photosystem II, enhanced levels of non-photochemical quenching (NPQ), accelerated dark-dependent decay rates of NPQ, elevated levels of open photosystem II complexes, and increased linear electron transfer rates following stress application compared to wild type plants. Embodiments of the invention may include increased biomass, which in some embodiments may be up to, and even greater than a 2-fold increase in above ground biomass yield in Fd2 transgenic plants compared to control plants.

Another aim of the invention may include the expression of a maize (Zea mays) mesophyll cell specific Fd1 in a C3 plant, such as an oil seed or oil crop. In this embodiment, a preferred oil crop may be Camelina sativa. Expression of this heterologous maize bundle sheath cell specific Fd1 gene may confer to a Camelina sativa plant enhanced tolerance to low temperature and high light stress as measured by reduced damage to photosystem II, enhanced levels of non-photochemical quenching (NPQ), accelerated dark-dependent decay rates of NPQ, elevated levels of open photosystem II complexes, and increased linear electron transfer rates following stress application compared to wild type plants. Embodiments of the invention may include increased biomass, which in some embodiments may be up to, and even greater than a 2-fold increase in above ground biomass yield in Fd2 transgenic plants compared to control plants.

Another aim of the invention may include the expression of a maize (Zea mays) mesophyll cell specific Fd1 in the chloroplasts of a C3 plant, such as Camelina sativa, wherein such expression may increase cyclic electron transfer rates and photosynthetic CO₂ fixation rates, which in some embodiments may be approximately 25% or more resulting in as much as a 2-fold increase in biomass accumulation in the transgenic plant. In another preferred embodiment, expression or overexpression of a maize Fd1 gene encoding the mesophyll cell specific ferredoxin in the chloroplasts of a C3 plant, such as Camelina sativa, wherein such expression may increase cold and heat stress tolerance of the photosynthetic apparatus including the level of NPQ as well as accelerating its rate of decay in the dark increasing the efficiency of photon utilization for photosynthesis. Additional embodiments may incorporate genetically modifying food crop plants to exhibit one or more enhanced characteristics as generally described herein.

Another aim of the invention may include the expression of a maize (Zea mays) Fd2 gene encoding the bundle sheath cell specific ferredoxin in the chloroplasts of a C3 plant, such as Camelina sativa, wherein such expression may increase linear electron transfer rates and photosynthetic CO₂ fixation rates, which in some embodiments may be approximately 25% or more resulting in as much as a 2-fold increase in biomass accumulation in the transgenic plant. In another preferred embodiment, expression or overexpression of a maize Fd2 gene encoding the bundle sheath cell specific ferredoxin in the chloroplasts of a C3 plant, such as Camelina sativa, wherein such expression may increase cold and heat stress tolerance of the photosynthetic apparatus including the level of NPQ as well as accelerating its rate of decay in the dark increasing the efficiency of photon utilization for photosynthesis.

Another aim of the invention may include the generation of genetically modifying a C3 food crop that express a heterologous photosynthetic Fd from a C4 plant that exhibits one or more enhanced characteristics as generally described herein. Another aim of the invention may include the generation of genetically modifying a C3 food crop plants that express a heterologous C4 photosynthetic Fd1 and/or Fd2 that exhibits one or more enhanced characteristics as generally described herein.

Another embodiment provides for use of a construct comprising one or more nucleic acids encoding a Fd1 and/or Fd2 protein from a C4 plant for: 1) making a transgenic C4 plant; 2) enhancing photosynthetic rates in a C3 plant; 3) enhancing either CET and/or LET photosynthetic electron transfer in a C3 plant; 4) enhancing the rate of photosynthetic CO₂ fixation in a C3 plant; 5) enhancing yield and/or biomass in a C3 plant; and 6) enhancing abiotic stress resistance in a C3 plant.

Additional aims of the invention may include one or more of the following embodiment:

1. A transgenic C3 plant expressing a heterologous polynucleotide sequence operably linked to a promoter sequence encoding at least one of the following:
photosynthetic ferredoxin-1 (Fd1) protein that enhances linear electron transport (LET) in said transgenic C3 plant;
photosynthetic ferredoxin-2 (Fd2) protein that enhances photosynthetic linear electron transport (LET) in said transgenic C3 plant; and
a combination of said photosynthetic Fd1 and Fd2 proteins.
   2. The transgenic C3 plant of embodiment 1 wherein said photosynthetic Fd1 protein is from a C4 plant and further comprises a mesophyll cell specific photosynthetic Fd1 protein from a C4 plant.
   3. The transgenic C3 plant of embodiment 2 wherein said photosynthetic Fd1 protein from a C4 plant is selected from the group consisting of: SEQ ID NO. 2, SEQ ID NO. 3, and an Fd1 variant thereof.
   4. The transgenic C3 plant of embodiment 2 wherein said heterologous nucleotide sequence encoding a C4 photosynthetic Fd1 protein is selected from the group consisting of: SEQ ID NO 6, SEQ ID NO. 7, and a nucleotide sequence having 85% sequence identity with at least one of said nucleotide sequences.
   5. The transgenic C3 plant of embodiment 3 wherein said photosynthetic Fd1 protein enhances photosynthetic cyclic electron transport (CET) in said transgenic C3 plant.
   6. The transgenic C3 plant of embodiment 1 wherein said photosynthetic Fd2 protein is from a C4 plant and further comprises a bundle sheath cell specific Fd2 protein from a C4 plant.
   7. The transgenic C3 plant of embodiment 6 wherein said photosynthetic Fd2 protein from a C4 plant is selected from the group consisting of: SEQ ID NO. 1, or an Fd2 variant thereof.
   8. The transgenic C3 plant of embodiment 6 wherein said heterologous nucleotide sequence encoding a C4 photosynthetic Fd2 protein is selected from the group consisting of: SEQ ID NO 4, SEQ ID NO. 5, and a nucleotide sequence having 85% sequence identity with at least one of said nucleotide sequences.
   9. The transgenic C3 plant of embodiment 7 wherein said photosynthetic Fd2 protein enhances photosynthetic linear electron transport (LET) in said transgenic C3 plant.
   10. The transgenic C3 plant of embodiment 1 wherein said transgenic C3 plant is selected from the group consisting of: a C3 oil seed crop, a C3 oil crop, and a C3 food crop.
   11. The transgenic C3 plant of embodiment 1 wherein said transgenic C3 plant is selected from the group consisting of: Cannabis, and hemp.
   12. A transgenic plant expressing a heterologous polynucleotide sequence encoding a photosynthetic ferredoxin (Fd) protein operably linked to a promoter sequence.
   13. The transgenic plant of embodiment 12 wherein said transgenic plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   14. The transgenic plant of embodiment 13 wherein said transgenic plant is a C3 plant.
   15. The transgenic plant of embodiment 14 wherein said photosynthetic Fd protein is a photosynthetic Fd protein from a C4 plant.
   16. The transgenic plant of embodiment 15 wherein the C4 plant is selected from the group consisting of selected from the group consisting of: a C4 plant from the genera Panicum, a C4 plant from the genera Saccharum, a C4 plant from the genera Setaria, a C4 plant from the genera sorghum and a C4 plant from the genera Zea.
   17. The transgenic plant of embodiment 15 wherein said photosynthetic Fd protein from a C4 plant is selected from the group consisting of: a bundle sheath cell specific photosynthetic Fd protein from a C4 plant, and mesophyll cell specific photosynthetic Fd protein from a C4 plant.
   18. The transgenic plant of embodiment 17 wherein said photosynthetic Fd protein from a C4 plant is selected from the group consisting of: photosynthetic ferredoxin-1 (Fd1) protein from a C4 plant, and photosynthetic ferredoxin-2 (Fd2) protein from a C4 plant.
   19. The transgenic plant of embodiment 18 wherein said photosynthetic Fd1 protein is from maize (Zea mays).
   20. The transgenic plant of embodiment 18 wherein said photosynthetic Fd2 protein is from maize (Zea mays).
   21. The transgenic plant of embodiment 18 wherein said photosynthetic Fd2 protein enhances linear electron transfer rates in said transgenic C3 plant.
   22. The transgenic plant of embodiment 18 wherein said photosynthetic Fd1 protein enhances photosynthetic cyclic electron transport (CET) in said transgenic C3 plant.
   23. The transgenic plant of embodiment 18 wherein said Fd1 protein has an amino acid sequence with at least 85% identity to an amino acid sequence selected from the group consisting of: SEQ ID NO. 2, and SEQ ID NO. 3.
   24. The transgenic plant of embodiment 18 wherein said Fd2 protein has an amino acid sequence with at least 85% identity to an amino acid sequence according to SEQ ID NO. 1.
   25. The transgenic plant of embodiment 1 wherein said heterologous polynucleotide sequence encoding a photosynthetic Fd protein comprises a heterologous polynucleotide sequence encoding photosynthetic Fd protein from a C4 plant.
   26. The transgenic plant of embodiment 25 wherein said a heterologous polynucleotide sequence encoding photosynthetic Fd protein from a C4 plant comprises a nucleic acid sequence selected from the group consisting of: SEQ ID NOs. 4, 5, 6, 7, and a nucleotide sequence having 85% sequence identity with at least one of said nucleotide sequences.
   27. The transgenic plant of embodiment 15 wherein said photosynthetic Fd protein has an amino acid sequence with at least 85% identity to an amino acid sequence selected from the group consisting of: SEQ ID NO. 8, SEQ ID NO. 9, and SEQ ID NO. 10.
   28. The transgenic plant of embodiment 14 wherein said transgenic C3 plant is a stably transformed transgenic C3 plant.
   29. The transgenic plant of embodiment 14 wherein said transgenic C3 plant is transformed through Agrobacterium-mediated transformation.
   30. The transgenic plant of embodiment 14 wherein said C3 plant is selected from the group consisting of: a C3 oilseed crop, a C3 oil crop, and a C3 food crop.
   31. The transgenic plant of embodiment 14 wherein said transgenic C3 plant is a Camelina sativa plant.
   32. The transgenic plant of embodiment 14 wherein said transgenic C3 plant is selected from the group consisting of: Cannabis, and hemp.
   33. A transformed seed of the transgenic C3 plant of embodiment 15.
   34. A transgenic C3 plant expressing a heterologous nucleotide sequence encoding a photosynthetic ferredoxin (Fd) protein from a C4 plant wherein said transgenic C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   35. The transgenic C3 plant of embodiment 34 wherein said photosynthetic Fd protein from a C4 plant is selected from the group consisting of: a bundle sheath cell specific photosynthetic Fd protein from a C4 plant, and mesophyll cell specific photosynthetic Fd protein from a C4 plant.
   36. The transgenic C3 plant of embodiment 34 wherein said photosynthetic Fd protein from a C4 plant comprises a protein is selected from the group consisting of: SEQ ID NOs. 1, 2, 3, 8, 9, 10, and Fd variants thereof.
   37. The transgenic C3 plant of embodiment 34 wherein said heterologous nucleotide sequence encoding a C4 photosynthetic Fd protein comprises a nucleotide sequence selected from the group consisting of: SEQ ID NOs. 4, 5, 6, 7, and a nucleotide sequence having 85% sequence identity with at least one of said nucleotide sequences.
   38. The transgenic C3 plant of embodiment 34 wherein said photosynthetic Fd protein from a C4 plant is a photosynthetic ferredoxin-1 (Fd1) protein from a C4 plant selected from the group consisting of: SEQ ID NO. 2, SEQ ID NO. 3, and an Fd1 variant thereof.
   39. The transgenic C3 plant of embodiment 34 wherein said heterologous nucleotide sequence encoding a C4 photosynthetic Fd protein comprises a heterologous nucleotide sequence encoding a C4 photosynthetic Fd1 protein selected from the group consisting of: SEQ ID NO. 6, SEQ ID NO. 7, and a nucleotide sequence having 85% sequence identity with at least one of said nucleotide sequences.
   40. The transgenic C3 plant of embodiment 34 wherein said photosynthetic Fd protein from a C4 plant is a photosynthetic ferredoxin-2 (Fd2) protein from a C4 plant according to SEQ ID NO. 1, or an Fd2 variant thereof.
   41. The transgenic C3 plant of embodiment 34 wherein said heterologous nucleotide sequence encoding a C4 photosynthetic Fd protein comprises a heterologous nucleotide sequence encoding a C4 photosynthetic Fd2 protein selected from the group consisting of: SEQ ID NO. 4, SEQ ID NO. 5, and a nucleotide sequence having 85% sequence identity with at least one of said nucleotide sequences.
   42. The transgenic C3 plant of embodiment 38 wherein said photosynthetic Fd1 protein enhances photosynthetic cyclic electron transport (CET) in said transgenic C3 plant.
   43. The transgenic C3 plant of embodiment 40 wherein said photosynthetic Fd2 protein enhances photosynthetic linear electron transport (LET) in said transgenic C3 plant.
   44. The transgenic C3 plant of embodiment 34 wherein said photosynthetic Fd protein from a C4 plant is selected from a group consisting of: photosynthetic Fd1 protein from Zea mays, and photosynthetic Fd2 protein from Zea mays.
   45. The transgenic C3 plant of embodiment 34 wherein said transgenic C3 plant is selected from the group consisting of: a C3 oil seed crop, a C3 oil crop, and a C3 food crop.
   46. The transgenic C3 plant of embodiment 34 wherein said transgenic C3 plant is a Camelina sativa plant.
   47. The transgenic C3 plant of embodiment 34 wherein said transgenic C3 plant is selected from the group consisting of: Cannabis, and hemp.
   48. The transgenic C3 plant of embodiment 34 wherein said transgenic C3 plant is transformed through Agrobacterium-mediated transformation.
   49. A transformed seed of the transgenic C3 plant of embodiment 34.
   50. A transgenic C3 plant cell expressing a heterologous nucleotide sequence encoding a photosynthetic ferredoxin (Fd) protein from a C4 plant.
   51. The transgenic C3 plant cell of embodiment 50 wherein said photosynthetic Fd protein from a C4 plant is selected from the group consisting of: a bundle sheath cell specific photosynthetic Fd protein from a C4 plant, and mesophyll cell specific photosynthetic Fd protein from a C4 plant.
   52. The transgenic C3 plant cell of embodiment 50 wherein said photosynthetic Fd protein from a C4 plant comprises a protein selected from the group consisting of: SEQ ID NOs. 1, 2, 3, 8, 9, 10, and Fd variant thereof.
   53. The transgenic C3 plant cell of embodiment 50 wherein said heterologous nucleotide sequence encoding a C4 photosynthetic Fd protein comprises a nucleotide sequence selected from the group consisting of: SEQ ID NOs. 4, 5, 6, 7, and a nucleotide sequence having 85% sequence identity with at least one of said nucleotide sequences.
   54. The transgenic C3 plant cell of embodiment 50 wherein said photosynthetic Fd protein from a C4 plant is a photosynthetic ferredoxin-1 (Fd1) protein from a C4 plant selected from the group consisting of: SEQ ID NO. 2, SEQ ID NO. 3, and Fd1 variant thereof.
   55. The transgenic C3 plant cell of embodiment 50 wherein said heterologous nucleotide sequence encoding a photosynthetic Fd protein from a C4 plant comprises a heterologous nucleotide sequence encoding a C4 photosynthetic Fd1 protein selected from the group consisting of: SEQ ID NO. 6, SEQ ID NO. 7, and a nucleotide sequence having 85% sequence identity with at least one of said nucleotide sequences.
   56. The transgenic C3 plant cell of embodiment 54 wherein said photosynthetic Fd1 protein enhances photosynthetic cyclic electron transport (CET) in said transgenic C3 plant.
   57. The transgenic C3 plant cell of embodiment 50 wherein said photosynthetic Fd protein from a C4 plant is a photosynthetic ferredoxin-2 (Fd2) protein from a C4 plant selected from the group consisting of: SEQ ID NO. 1, and an Fd2 variant thereof.
   58. The transgenic C3 plant cell of embodiment 50 wherein said heterologous nucleotide sequence encoding a photosynthetic Fd protein from a C4 plant comprises a heterologous nucleotide sequence encoding a C4 photosynthetic Fd2 protein selected from the group consisting of: SEQ ID NO. 4, SEQ ID NO. 5, and a nucleotide sequence having 85% sequence identity with at least one of said nucleotide sequences.
   59. The transgenic C3 plant cell of embodiment 57 wherein said photosynthetic Fd2 protein enhances photosynthetic linear electron transport (LET) in said transgenic C3 plant.
   60. The transgenic C3 plant cell of embodiment 50 wherein said photosynthetic Fd protein from a C4 plant is selected from a group consisting of: a photosynthetic Fd1 protein from Zea mays, and photosynthetic Fd2 protein from Zea mays.
   61. The transgenic C3 plant cell of embodiment 50 wherein said transgenic C3 plant cell is selected from the group consisting of: a C3 plant cell from an oil seed crop, a C3 plant cell from an oil crop, and a C3 plant cell from a food crop.
   62. The transgenic C3 plant cell of embodiment 50 wherein said transgenic C3 plant cell is a Camelina sativa plant.
   63. The transgenic C3 plant cell of embodiment 50 wherein said transgenic C3 plant cell is selected from the group consisting of: Cannabis, and hemp.
   64. The transgenic C3 plant cell of embodiment 50 wherein said transgenic C3 plant cell is in a suspension cell culture.
   65. The transgenic C3 plant cell of embodiment 50 wherein said transgenic C3 plant cell is transformed through Agrobacterium-mediated transformation.
   66. A transgenic C3 plant expressing a heterologous nucleotide sequence encoding a photosynthetic ferredoxin-1 (Fd1) protein from a C4 plant wherein said transgenic C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   67. The transgenic C3 plant of embodiment 66 wherein said photosynthetic Fd1 protein from a C4 plant comprises a mesophyll cell specific photosynthetic Fd1 protein from a C4 plant.
   68. The transgenic C3 plant of embodiment 67 wherein said photosynthetic Fd1 protein from a C4 plant is selected from the group consisting of: SEQ ID NO. 2, SEQ ID NO. 3, and an Fd1 variant thereof.
   69. The transgenic C3 plant of embodiment 66 wherein said heterologous nucleotide sequence encoding a C4 photosynthetic Fd1 protein is selected from the group consisting of: SEQ ID NO. 6, SEQ ID NO. 7, and a nucleotide sequence having 85% sequence identity with at least one of said nucleotide sequences.
   70. The transgenic C3 plant of embodiment 68 wherein said photosynthetic Fd1 protein enhances photosynthetic cyclic electron transport (CET) in said transgenic C3 plant.
   71. The transgenic C3 plant of embodiment 68 wherein said photosynthetic Fd1 protein from a C4 plant comprises a photosynthetic Fd1 protein from Zea mays.
   72. The transgenic C3 plant of embodiment 66 wherein said transgenic C3 plant is selected from the group consisting of: a C3 oil seed crop, a C3 oil crop, and a C3 food crop.
   73. The transgenic C3 plant of embodiment 66 wherein said transgenic C3 plant is a Camelina sativa plant.
   74. The transgenic C3 plant of embodiment 66 wherein said transgenic C3 plant is selected from the group consisting of: Cannabis, and hemp.
   75. The transgenic C3 plant of embodiment 66 wherein said transgenic C3 plant is transformed through Agrobacterium-mediated transformation.
   76. The transgenic C3 plant of embodiment 66 and further comprising the step of expressing a heterologous nucleotide sequence encoding a photosynthetic ferredoxin-2 (Fd2) protein from a C4 plant in said transgenic plant.
   77. The transgenic C3 plant of embodiment 76 wherein said photosynthetic Fd2 protein from a C4 plant is selected from the group consisting of: SEQ ID NO. 1, or an Fd2 variant thereof.
   78. A transformed seed of the transgenic C3 plant of embodiment 66.
   79. A transgenic C3 plant expressing a heterologous nucleotide sequence encoding a photosynthetic ferredoxin-2 (Fd2) protein from a C4 plant wherein said transgenic C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   80. The transgenic C3 plant of embodiment 79 wherein said photosynthetic Fd2 protein from a C4 plant comprises a bundle sheath cell specific Fd2 protein from a C4 plant. 81. The transgenic C3 plant of embodiment 79 wherein said photosynthetic Fd2 protein from a C4 plant is selected from the group consisting of: SEQ ID NO. 1, or an Fd2 variant thereof.
   82. The transgenic C3 plant of embodiment 79 wherein said heterologous nucleotide sequence encoding a C4 photosynthetic Fd2 protein is selected from the group consisting of: SEQ ID NO. 4, SEQ ID NO. 5, and a nucleotide sequence having 85% sequence identity with at least one of said nucleotide sequences.
   83. The transgenic C3 plant of embodiment 81 wherein said photosynthetic Fd2 protein enhances photosynthetic linear electron transport (LET) in said transgenic C3 plant.
   84. The transgenic C3 plant of embodiment 81 wherein said photosynthetic Fd2 protein from a C4 plant comprises a photosynthetic Fd2 protein from Zea mays.
   85. The transgenic C3 plant of embodiment 79 wherein said transgenic C3 plant is selected from the group consisting of: a C3 oil seed crop, a C3 oil crop, and a C3 food crop.
   86. The transgenic C3 plant of embodiment 79 wherein said transgenic C3 plant is a Camelina sativa plant.
   87. The transgenic C3 plant of embodiment 79 wherein said transgenic C3 plant is selected from the group consisting of: Cannabis, and hemp.
   88. The transgenic C3 plant of embodiment 79 wherein said transgenic C3 plant is transformed through Agrobacterium-mediated transformation.
   89. The transgenic C3 plant of embodiment 79 and further comprising the step of expressing a heterologous nucleotide sequence encoding a photosynthetic ferredoxin-1 (Fd1) protein from a C4 plant in said transgenic plant.
   90. The transgenic C3 plant of embodiment 89 wherein said photosynthetic Fd1 protein from a C4 plant is selected from the group consisting of: SEQ ID NO. 2, SEQ ID NO. 3, and variants thereof.
   91. A transformed seed of the transgenic C3 plant of embodiment 79.
   92. A method of enhancing photosynthesis comprising the step of transforming a C3 plant by introducing an expression cassette comprising a heterologous polynucleotide sequence operably linked to a promoter sequence encoding at least one of the following:
photosynthetic ferredoxin-1 (Fd1) protein from a C4 plant that enhances linear electron transport (LET) in said transgenic C3 plant; and
photosynthetic ferredoxin-2 (Fd2) protein from a C4 plant that enhances photosynthetic linear electron transport (LET) in said transgenic C3 plant.
   93. The method of embodiment 92 wherein said photosynthetic Fd2 protein comprises a photosynthetic Fd2 protein selected from the group consisting of: an amino acid sequence according to SEQ ID NO. 1, and an Fd2 variant thereof.
   94. The method of embodiment 92 wherein said photosynthetic Fd1 protein comprises a photosynthetic Fd1 protein selected from the group consisting of: an amino acid sequence according to SEQ ID NO. 2, an amino acid sequence according to SEQ ID NO. 3, and an Fd1 variant thereof.
   95. The method of embodiment 92 wherein said photosynthetic Fd1 sequence comprises a polynucleotide sequence selected from the group consisting of: SEQ ID NO. 6, SEQ ID NO. 7, or a polynucleotide having at least 85% sequence identity to SEQ ID NO. 6, or SEQ ID NO. 7.
   96. The method of embodiment 94 wherein said transformed C3 plant is selected from the group consisting of: a C3 oil seed crop, a C3 oil crop, and a C3 food crop.
   97. The method of embodiment 94 wherein said transformed C3 plant is selected from the group consisting of: Cannabis, and hemp.
   98. The method of embodiment 92 wherein said transformed C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   99. A method of enhancing photosynthesis comprising:
transforming a C3 plant by introducing an expression cassette comprising a heterologous polynucleotide sequence that encodes a photosynthetic ferredoxin (Fd) protein from a C4 plant operably linked to a promoter sequence.
   100. The method of embodiment 99 wherein said photosynthetic Fd sequence comprises a polynucleotide sequence selected from the group consisting of: SEQ ID NO. 4-7, or a polynucleotide having at least 85% sequence identity to at least one polynucleotide of SEQ ID NO. 4-7.
   101. The method of embodiment 99 wherein said photosynthetic Fd protein comprises a photosynthetic Fd protein selected from the group consisting of: a ferredoxin-1 (Fd1) protein, a ferredoxin-2 protein (Fd2), and Fd1 and Fd2 variants thereof.
   102. The method of embodiment 101 wherein said photosynthetic Fd2 protein enhances photosynthetic linear electron transfer rates is said transformed C3 plant.
   103. The method of embodiment 101 wherein said photosynthetic Fd1 protein enhances photosynthetic cyclic electron transfer rates is said transformed C3 plant.
   104. The method of embodiment 101 wherein said photosynthetic Fd1 protein comprises a photosynthetic Fd1 protein selected from the group consisting of: an amino acid sequence according to SEQ ID NO. 2, an amino acid sequence according to SEQ ID NO. 3, and an Fd1 variant thereof.
   105. The method of embodiment 101 wherein said photosynthetic Fd2 protein comprises a photosynthetic Fd2 protein selected from the group consisting of: an amino acid sequence according to SEQ ID NO. 1, and an Fd2 variant thereof.
   106. The method of embodiment 99 wherein said photosynthetic Fd protein comprises a photosynthetic Fd protein selected from the group consisting of: an amino acid sequence according to SEQ ID NO. 8-10, and, and an Fd variant thereof.
   107. The method of embodiment 99 wherein said transformed C3 plant is selected from the group consisting of: a C3 oil seed crop, a C3 oil crop, and a C3 food crop.
   108. The method of embodiment 99 wherein said transformed C3 plant is selected from the group consisting of: Cannabis, and hemp.
   109. The method of embodiment 99 wherein said C3 plant is transformed through Agrobacterium-mediated transformation.
   110. The method of embodiment 101 wherein said transformed C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   111. The method of embodiment 99 wherein said C3 plant is stably transformed.
   112. A transformed plant cell from said transformed C3 plant of embodiment 110.
   113. A transformed seed from said transformed C3 plant of embodiment 110.
   114. A method of enhancing photosynthesis comprising:
expressing in a C3 plant a heterologous polynucleotide sequence encoding a photosynthetic ferredoxin-1 (Fd1) protein from a C4 plant operably linked to a promoter sequence.
   115. The method of embodiment 114 wherein said photosynthetic Fd1 protein enhances cyclic electron transfer rates is said transformed C3 plant.
   116. The method of embodiment 115 wherein said photosynthetic Fd1 protein comprises a photosynthetic Fd1 protein selected from the group consisting of: an amino acid sequence according to SEQ ID NO. 2, an amino acid sequence according to SEQ ID NO. 3, and an Fd1 variant thereof.
   117. The method of embodiment 114 wherein said photosynthetic Fd1 sequence comprises a polynucleotide sequence selected from the group consisting of: SEQ ID NO. 6, SEQ ID NO. 7, or a polynucleotide having at least 85% sequence identity to SEQ ID NO. 6, or SEQ ID NO. 7.
   118. The method of embodiment 116 wherein said transformed C3 plant is selected from the group consisting of: a C3 oil seed crop, a C3 oil crop, and a C3 food crop.
   119. The method of embodiment 116 wherein said transformed C3 plant is selected from the group consisting of: Cannabis, and hemp.
   120. The method of embodiment 117 wherein said C3 plant is transformed through Agrobacterium-mediated transformation.
   121. The method of embodiment 115 wherein said transformed C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   122. The method of embodiment 115 wherein said C3 plant is stably transformed.
   123. A transformed plant cell from said transformed C3 plant of embodiment 121.
   124. A transformed seed from said transformed C3 plant of embodiment 121.
   125. A method of enhancing photosynthesis comprising:
expressing in a C3 plant a heterologous polynucleotide sequence encoding a photosynthetic ferredoxin-2 (Fd2) protein from a C4 plant operably linked to a promoter sequence.
   126. The method of embodiment 125 wherein said photosynthetic Fd2 protein enhances linear electron transfer rates is said transformed C3 plant.
   127. The method of embodiment 126 wherein said photosynthetic Fd2 protein comprises a photosynthetic Fd2 protein selected from the group consisting of: an amino acid sequence according to SEQ ID NO. 1, and an Fd2 variant thereof.
   128. The method of embodiment 126 wherein said photosynthetic Fd2 sequence comprises a polynucleotide sequence selected from the group consisting of: SEQ ID NO. 4, SEQ ID NO. 5, or a polynucleotide having at least 85% sequence identity to at least one polynucleotide of SEQ ID NO. 4, or SEQ ID NO. 5.
   129. The method of embodiment 127 wherein said transformed C3 plant is selected from the group consisting of: a C3 oil seed crop, a C3 oil crop, and a C3 food crop.
   130. The method of embodiment 127 wherein said transformed C3 plant is selected from the group consisting of: Cannabis, and hemp.
   131. The method of embodiment 127 wherein said C3 plant is transformed through Agrobacterium-mediated transformation.
   132. The method of embodiment 126 wherein said transformed C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   133. The method of embodiment 126 wherein said C3 plant is stably transformed.
   134. A transformed plant cell from said transformed C3 plant of embodiment 132.
   135. A transformed seed from said transformed C3 plant of embodiment 132.
   136. An expression cassette for the expression of at least one C4 ferredoxin protein in a C3 plant comprising in operable linkage:
a promoter that functions in a C3 plant cell, and
a nucleic acid sequence encoding a C4 ferredoxin (Fd) protein.
   137. The expression cassette of embodiment 136 wherein said C4 Fd protein is selected from the group consisting SEQ ID NOs. 8-10.
   138. A vector comprising the expression cassette of embodiment 136 or 137.
   139. A transformed plant comprising the expression cassette of embodiment 136 or 137.
   140. The transformed plant of embodiment 139 wherein said expression cassette is incorporated into the plant or plant cell genome.
   141. The transformed plant of embodiment 139 or 140 wherein said transformed plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.
   142. An expression cassette for the expression of at least one C4 ferredoxin protein comprising in operable linkage:
a promoter that functions in a C3 plant cell, and
a nucleic acid sequence encoding a C4 ferredoxin (Fd) protein with at least 85% identity to an amino acid sequence selected from the group consisting of SEQ ID NOs: 4-7.
   143. The expression cassette of embodiment 142 wherein said C4 Fd protein is selected from the group consisting SEQ ID NOs. 1-3.
   144. A vector comprising the expression cassette of embodiment 142 or 143.
   145. A transformed plant comprising the expression cassette of embodiment 142 or 143.
   146. The transformed plant of embodiment 145 wherein said expression cassette incorporated into the plant or plant cell genome.
   147. The transformed plant of embodiment 145 or 146 wherein said transformed plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.

As detailed below, such enhanced characteristics could not have been anticipated or expected by those of ordinary skill in the art since in maize bundle sheath cells, Fd2 increases cyclic electron transfer (CET) and not linear electron transfer (LET) rates. Furthermore, Fd2 has reduced affinity and catalytic turnover rates for ferredoxin NADP reductase compared to the maize Fd1 protein which enhances linear electron transfer. Additionally, mesophyll cell specific Fd1 increases linear electron transfer (LET) rates and not cyclic electron transfer (CET). However, as shown herein, when a C4 photosynthetic Fd1 and/or Fd2 are expressed in a C3 plant, their roles are reversed such that Fd2 increases LET rates and not CET rates, while Fd1 increases CET rates and not LET rates. Indeed, such observations render the inventive technology not only novel, but counter to the expectations and understanding of those skilled in the art.

Further scope of the applicability of the presently disclosed embodiments will become apparent from the detailed description and drawing(s) provided below. However, it should be understood that the detailed description and specific examples, while indicating preferred embodiments of this disclosure, are given by way of illustration only since various changes and modifications within the spirit and scope of these embodiments will become apparent to those skilled in the art from this detailed description.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1A-C: Expression of Maize Fd2 in Camelina Stevia. (A) Example of a phenotypic observation of an overexpressing CaMV 35S:FD2 line; (B) Chlorophyll measurements of four CaMV 35S: FD2 transgenic lines; and (C) Reverse transcriptase-PCR (RT-PCR) analysis demonstrating expression levels of the four CaMV 35S:FD2 transgenic lines in one embodiment thereof.

FIG. 2A-C: (A) Exemplary cloning overview of the FD2 and FD1 genes; (B) expression vectors including the FD2 gene in one embodiment thereof; and (C) expression vectors including the FD1 gene in one embodiment thereof.

FIG. 3A-D: Gas exchange measurements of CaMV 35S:FD2 high overexpressing lines under greenhouse conditions: (A) gas exchange measurements of photosynthesis (μmol CO₂ m-2a-I); (B) measurements of internal leaf CO2 concentrations, Ci (μmol CO₂ m-I); (C) gas exchange measurements of stomatal conductance (μmol H₂O m-2a-I); and (D) gas exchange measurements of transpiration rate (μmol H₂O m-2a-I).

FIG. 4: Gas exchange measurements of CaMV 35S:FD2 high overexpressing lines under field conditions: (A) photosynthetic CO₂ assimilation; (B) intercellular CO₂ concentration; (C) stomatal conductance; and (D) transpiration rate.

FIG. 5: Characterization of plant size and/or seed weight of CaMV 35S:FD2 high overexpressing lines under field conditions: (A) average plant size+seed weight (g); and (B) average seed weight (g).

FIG. 6: Characterization of excitation energy distribution (A) in wild-type line; and (B) characterization of excitation energy distribution in CaMV 35S:FD2 overexpression line.

FIG. 7: Characterization of the effect of chilling or high light stress on maximal photochemical efficiency of PSII in one embodiment thereof.

FIG. 8: (A) Characterization of non-photochemical quenching (NPQ) in WT and Fd2 leaves as under control (non-stress) conditions; (B) characterization of the effect of chilling on non-photochemical quenching (NPQ) in WT and Fd2 leaves; and (C) Characterization of the effect of high temperature+high light (HT+HL) stress on non-photochemical quenching (NPQ) in WT and Fd2 leaves.

FIG. 9: (A) Characterization of linear electron transport rate (ETR) under control conditions; (B) characterization of the effect of chilling on linear electron transport rate (ETR); and (C) characterization of the effect of high light stress on linear electron transport rate (ETR).

FIG. 10: Diagram of Fd and FNR to photosynthetic electron transport in the bundle sheath cell chloroplasts of maize (maize mesophyll chloroplast; and (B) maize bundle sheath chloroplast.

FIG. 11: Exemplary maize FD1 and FD2 sequences and alignments. The amino acid sequence encoded by the open reading frame is shown below the cDNA nucleotide sequence. The determined N-terminal amino acid sequence and C-terminal residue of the mature form of Fd2 are underlined. C, The amino acid sequence of maize Fd2 d is compared with that of maize Fd1. Gaps, denoted by dashes, have been inserted to achieve maximum homology. Identical amino acid residues between Fd1 and Fd2 are indicated by white letters on a black background.

FIG. 12: Demonstrates immunoblot analyses of ferredoxin (FD) proteins content in FD1 and/or FD2 overexpression Camelina lines expressing Maize Fd proteins.

FIG. 13A-D: Demonstrates P700 oxidation and reduction kinetics in (A) Camelina expressing maize FD1 lines and (B) Camelina expressing maize FD2 lines.

FIG. 14A-D: Demonstrates P700 oxidation and reduction kinetics in DCMU treated Camelina expressing maize FD1 (A) and Camelina expressing maize FD2 (B) overexpression lines.

FIG. 15A-B: Demonstrates chlorophyll fluorescence Fo levels increase during a light to dark transition in FD1 (A) and FD2 (B) overexpression lines.

FIG. 16A-D: Demonstrates alterations in electron transport rates (ETR) in FD overexpression lines.

FIG. 17A-B: Demonstrates alterations in non-photochemical quenching (NPQ) induction in FD1 (A) and FD2 (B) overexpression lines.

FIG. 18A-B: Demonstrates CO₂ gas exchange measurement of greenhouse grown plants. Each data point represents the average 3 to 6 of values on independent plants, and error bars represent SD of 3 to 6 technical replicates.

FIG. 19A-D: Field trial measurement for Photosynthetic CO₂ gas exchange measurements for Fd2 transformants and 4-gene (algal bicarbonate transporter complex) construct (HLA3, PGR5, LCIA, BCA) under cloudy to partially sunny weather conditions. (A) photosynthesis; (B) stomatal conductance; (C) intercellular CO₂; and (D) transpiration rate.

FIG. 20A-D: Field trial measurement for Photosynthetic CO₂ gas exchange measurements for Fd2 transformants and 4-gene construct (HLA3, PGR5, LCIA, BCA) under sunny weather conditions. (A) photosynthesis; (B) stomatal conductance; (C) intercellular CO₂; and (D) transpiration rate.

FIG. 21A-B: Biomass and yield production from field trial (first harvest). (A) seed; and (B) plants+seeds.

FIG. 22: Biomass and yield production from field trial (second harvest). (A) seed; and (B) plants+seeds.

### MODE(S) FOR CARRYING OUT THE INVENTION(S)

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries. The present disclosure is explained in greater detail below. This disclosure is not intended to be a detailed catalog of all the different ways in which embodiments of this disclosure can be implemented, or all the features that can be added to the instant embodiments. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the scope of the instant disclosure. Hence, the following specification is intended to illustrate some particular embodiments of the disclosure, and not to exhaustively specify all permutations, combinations, and variations thereof.

The invention includes systems, methods, and compositions related to the enhancement of photosynthetic electron transfer rates, abiotic stress tolerance, CO₂ fixation rates, and associated increase in biomass in plants. These methods and associated transgenic plants encompass the expression or overexpression of one or more genes that improve photosynthetic electron transfer rates, abiotic stress tolerance, CO₂ fixation rates, and biomass in plants. Such enhanced plant characteristics may be achieved through the expression or overexpression of select ferredoxin coding sequences or sequences in a plant or plant cell. Methods of the invention include the manipulation of photosynthesis through expression of heterologous genes encoding proteins involved in photosynthesis. Specifically, the methods disclosed herein encompass any method of expressing a ferredoxin sequence from a C4 plant, or a variant thereof, in a C3 plant or cell. That is, any C3 plant may be transformed to express a heterologous C4 ferredoxin sequence, or the C4 ferredoxin sequence may be introduced into a C3 plant via a C4 ferredoxin expression construct. In one embodiment, the methods and compositions disclosed herein describe strategies to transform a C3 plant or call to express genes encoding C4 Fd protein, preferably a genes encoding an Fd1 and/or Fd2 protein from a C4 plant.

Preferred embodiments may include the manipulation of photosynthesis through expression of heterologous ferredoxin genes encoding proteins involved in photosynthesis. Specifically, the methods disclosed herein encompass any method of expressing a ferredoxin-1 (Fd2), or ferredoxin-2 (Fd2) sequence from a C4 plant, or a variant thereof, in a C3 plant or cell. That is, any C3 plant may be transformed to express a heterologous C4 Fd1 or Fd2 sequence, or the C4 Fd1 or Fd2 sequence may be introduced into a C3 plant via a C4 Fd1 or Fd2 expression construct. Through, expression of said C4 Fd1 or Fd2 proteins in a C3 plant or plant cell, the plant can have a resulting increase in photosynthetic electronic transfer, photosynthetic efficiency, plant growth rate, plant height, abiotic stress resistance, and/or plant yield/biomass.

The C4 photosynthetic Fd sequences disclosed herein can be any ferredoxin that contributes to the transport of electrons in a C4 photosynthesis process. For example, a photosynthetic Fd1 polynucleotide coding sequence according to SEQ ID NO. 6-7, may encode a ferredoxin protein as provided in SEQ ID NO: 2 and/or 2 respectively, and variants and fragments thereof having LET activity in a C4 plant. In another example, a photosynthetic Fd2 gene, provided in SEQ ID NO. 4-5, may encode a ferredoxin protein as provided in SEQ ID NO: 1, and variants and fragments thereof having CET activity in a C4 photosynthetic plant. Additional embodiments may include variant C4 photosynthetic Fd sequences, such as amino acid sequences identified in SEQ ID NO 8-10, that may be expressed in a C3 plant and generate one or more of the enhanced characteristics described generally herein.

The C4 photosynthetic Fd sequences disclosed herein can be any ferredoxin that exhibit photosynthetic electron transport in a C3 plant that is the opposite of its photosynthetic electron transport activity in a C4 plant, or that results in the enhanced characteristics generally described herein. For example, the FD1 polynucleotide coding sequence, sometimes interchangeable referred to as a gene, provided in SEQ ID NOs. 6 or 7, may encode a photosynthetic Fd protein, for example as identified in SEQ ID NOs. 2 or 3, having CET activity when expressed a C3 plant. In another example, the FD2 gene according to SEQ ID NOs. 4 or 5, may encode a photosynthetic Fd protein according to SEQ ID NO: 1, having LET activity when expressed in a C3 photosynthetic plant.

In one embodiment, C4 photosynthetic Fd sequences can be identified and/or isolated from any C4 photosynthetic organism, and may include variants, such as those identified in SEQ ID NOs. 8-10. For example, certain C4 Fd polynucleotide, sequences such as SEQ ID NOs. 4-7, and amino acid sequences SEQ ID NOs. 1-3, can be isolated from Z. mays.

In one preferred embodiment, the invention includes systems, methods, and compositions related to the enhancement of photosynthetic electron transfer rates, abiotic stress tolerance, CO₂ fixation rates, and associated increase in biomass in C3 plants. These methods and associated transgenic plants encompass the expression or overexpression of one or more genes from a C4 plant that improve photosynthetic electron transfer rates, abiotic stress tolerance, CO₂ fixation rates, and biomass in plants. Such enhanced plant characteristics may be achieved through the heterologous (stable or transient) expression or overexpression of select Fd polynucleotides and/or proteins, or variants thereof, from a C4 plant in a C3 plant or plant cell.

In one embodiment, the invention includes systems, methods, and compositions related to the enhancement of photosynthetic electron transfer rates, abiotic stress tolerance, CO₂ fixation rates, and associated increase in biomass in C3 plants through heterologous expression of a Fd1 coding sequence, or a variant thereof, from a C4 plant in said C3 plant or cell. In another preferred embodiment, the invention includes systems, methods, and compositions related to the enhancement of photosynthetic electron transfer rates, abiotic stress tolerance, CO₂ fixation rates, and associated increase in biomass in C3 plants through heterologous expression of Fd2 coding sequence, or a variant thereof, or a variant thereof, from a C4 plant in said C3 plant or cell.

In one embodiment, the invention includes systems, methods, and compositions related to the enhancement of photosynthetic electron transfer rates, abiotic stress tolerance, CO₂ fixation rates, and associated increase in biomass in C3 plants through heterologous expression of a an expression cassette encoding one or more polynucleotides selected from SEQ ID NO. 4-7, operably linked to a promoter in a C3 plant. In another preferred embodiment, the invention includes systems, methods, and compositions related to the enhancement of photosynthetic electron transfer rates, abiotic stress tolerance, CO₂ fixation rates, and associated increase in biomass in C3 plants through heterologous expression of one or more polypeptide selected from SEQ ID NO. 1-3, and 8-10, or variants thereof, in said C3 plant.

In one embodiment, the invention includes a stably transformed C3 plant or plant cell expressing one or more heterologous Fd1 and/or Fd2 polypeptides according to SEQ ID NO. 2-3, and SEQ ID NO. 1, respectively. In one preferred embodiment, a C3 plant or plant cell may be transformed to express one or more of said heterologous Fd polypeptides sequences. In this embodiment, a C3 plant or plant cell may be transformed with a heterologous Fd1 polynucleotide according to the sequence identified as SEQ ID NO. 6 or 7, or a variant thereof, and/or a heterologous Fd2 polynucleotide according to the sequence identified as SEQ ID NO. 4 or 5, or a variant thereof. In another embodiment, a C3 plant or plant cell may be transformed with a heterologous Fd1 polynucleotide encoding a polypeptide according to SEQ ID NO. 2 or 3, or a variant thereof, and/or a heterologous Fd2 polynucleotide encoding according to the sequence identified as SEQ ID NO. 1, or a variant thereof.

In one embodiment, the enhancement of photosynthetic electron transfer rates, CO₂ fixation rates, abiotic stress tolerance, and an increase in biomass may be achieved through the overexpression of the a bundle sheath cell-specific Fd2 protein from a C4 plant in the chloroplasts of a C3 plant, such as a food crop, an oil seed or oil crop plant, such as Camelina sativa. In one preferred embodiment, a C4 plant Fd2 gene according to SEQ ID NO. 4 or 5, or a variant thereof, may be expressed in a transgenic C3 plant. Additional embodiments may include expression or overexpression of a bundle sheath cell-specific Fd2 protein according to SEQ ID NO. 1, or a variant thereof, from a C4 plant in a select food crop. In this preferred embodiment, a select food crop plant may preferably be a C3-type plant.

In one embodiment, the enhancement of photosynthetic electron transfer rates, CO₂ fixation rates, abiotic stress tolerance, and an increase in biomass may be achieved through the overexpression of the a mesophyll cell specific Fd1 protein from a C4 plant in the chloroplasts of a C3 plant, such as a food crop, an oil seed, or oil crop plant such as Camelina sativa. In one preferred embodiment, a C4 plant Fd1 polynucleotide according to SEQ ID NO. 6 or 7, or a variant thereof, may be expressed in a transgenic C3 plant. Additional embodiments may include expression or overexpression of a mesophyll cell specific Fd1 protein according to SEQ ID NO. 2-3, or a variant thereof, from a C4 plant in a select food crop. In this preferred embodiment, a select food crop plant may preferably be a C3-type food crop.

In another embodiment, the present invention provides for a transgenic plant comprising within its genome, and expressing or overexpressing, a heterologous nucleotide sequence encoding a heterologous Fd2 coding sequence that may be expressed in the chloroplast. The heterologous Fd2 protein expressed in this transgenic or genetically modified plant may be selected from a C4 plant, such as a Zea mayes (Maize) plant. The Fd2 protein expressed in this transgenic or genetically modified plant may be selected from an Fd2 protein, identified as SEQ ID NO. 1, or a variant or homolog thereof. It should be noted that all protein sequences provided herein also encompass their corresponding nucleotide sequences and vice versa.

In another embodiment, the present invention provides for a transgenic C3 plant expressing or overexpressing a heterologous nucleotide sequence encoding an Fd1 protein. The Fd1 protein expressed in this transgenic or genetically modified C3 plant may be selected from an Fd1 nucleotide sequence; for example, the sequence identified SEQ ID NO. 2-3, or a variant or homolog thereof.

In another embodiment, the present invention provides for a transgenic C3 plant expressing or overexpressing a heterologous nucleotide sequence encoding a C4 photosynthetic Fd protein. The Fd protein expressed in this transgenic or genetically modified C3 plant may be selected from an Fd nucleotide sequence; for example, the sequence identified SEQ ID NO. 8-10, or a variant or homolog thereof.

In another embodiment, the present invention provides for a transgenic plant comprising within its genome, and expressing or overexpressing, a heterologous nucleotide sequence encoding a heterologous Fd1 coding sequence. The heterologous Fd1 protein expressed in this transgenic or genetically modified plant may be selected from a C4 plant, such as a Zea Mayes (Maize) plant. The Fd1 protein expressed in this transgenic or genetically modified plant may be selected from an Fd2 protein, identified as SEQ ID NO. 2 or 3, or a variant or homolog thereof. It should be noted that all protein sequences provided herein also encompass their corresponding nucleotide sequences and vice versa.

In another embodiment, the present invention provides for a transgenic C3 plant, such as a Camelina sativa plant, expressing and/or overexpressing a heterologous nucleotide sequence encoding an Fd1 and/or Fd2 protein. The Fd1 and/or Fd2 heterologous nucleotide(s) expressed in this transgenic or genetically modified Camelina sativa plant may be selected from an Fd2 nucleotide sequence identified as SEQ ID NO. 4 or 5, or a variant or homolog thereof, and/or an Fd1 nucleotide sequence identified as SEQ ID NO. 6 or 7. The Fd2 protein expressed in this transgenic or genetically modified Camelina sativa plant may be selected from an Fd2 protein identified as SEQ ID NO. 1, or a variant or homolog thereof, and/or an Fd1 protein identified as SEQ ID NO. 2-3, or a variant or homolog thereof.

In another embodiment, the present invention provides for a transgenic plant as herein described, where the Fd2 and/or Fd2 protein, and/or corresponding nucleotide sequence, has an amino acid/nucleotide sequence at least 70% identical/homology one another). Alternatively, the sequence identity/sequence similarity in some embodiments may be about 70%, 75%, 85%, 90%, 91%, 92%, 93%, 94%, 95%, 96%, 97%, 98%, 99%, and/or 100% to those specifically disclosed including variants and homologs.

In another embodiment, the invention provides systems and methods of making a transgenic plant as described herein. In a preferred embodiment, said method comprises expressing or overexpressing, in a C3 plant, such as Camelina sativa, a heterologous nucleotide sequences encoding an Fd2 and/or Fd1 protein or a variant or homolog thereof.

The transgenic plant of an embodiment disclosed herein may be a C3 plant, such as a transgenic such as Camelina sativa plant or a transgenic food crop plant such as rice (Oryza sativa), wheat (Triticum spp.), barley (Hordeum vulgare), rye (Secale cereale), and oat (Avena sativa); soybean (Gycine max), peanut (Arachis hypogaea), cotton (Gossypium spp.), sugar beets (Beta vulgaris), tobacco (Nicotiana tabacum), spinach (Spinacea oleracea), soybean (Glycine max), or potato (Solanum tuberosum). The heterologous nucleotide sequences are described in an embodiment may be codon optimized for expression in said transgenic plant. It should be noted that these plants are presented as non-limiting examples only.

One aspect of the present invention provides for a transgenic C3 plant expressing a heterologous Fd protein, such as Fd1 and/or Fd2, as described herein which exhibits enhanced CO₂ fixation and/or CO₂ fixation compared to an otherwise identical control plant grown under the same conditions, for example wherein CO₂ fixation may be enhanced in the range of from about 10% to about 50% compared to that of an otherwise identical control plant grown under the same conditions.

Another aspect of the present invention provides for a transgenic C3 plant expressing a heterologous photosynthetic Fd protein, such as Fd1 and/or Fd2, as described herein which exhibits enhanced photosynthetic electron transfer rates compared to an otherwise identical control plant grown under the same conditions, for example, wherein enhanced photosynthetic electron transfer rates may be enhanced in the range of from about 10% to about 50% compared to that of an otherwise identical control plant grown under the same conditions.

Another aspect of the present invention provides for a transgenic C3 plant expressing a heterologous photosynthetic Fd protein, such as Fd1 and/or Fd2, as described herein which exhibits enhanced biomass accumulation compared to an otherwise identical control plant grown under the same conditions, for example wherein biomass accumulation may be enhanced in the range of from about 1- to 3-fold compared to that of an otherwise identical control plant grown under the same conditions.

Another aspect of the present invention provides for a transgenic C3 plant expressing a heterologous photosynthetic Fd protein, such as Fd1 and/or Fd2, as described herein which exhibits enhanced abiotic tolerance compared to an otherwise identical control plant grown under the same conditions. For example, in a select transgenic strain, cold and heat stress tolerance may be enhanced in of the photosynthetic apparatus including the level of non-photochemical quenching (NPQ) as well as accelerating its rate of decay in the dark increasing the efficiency of photon utilization for photosynthesis, elevated levels of open photosystem II complexes, and increased linear electron transfer rates following stress application compared to wild type plants grown under the same conditions.

One embodiment of the present invention provides for a transgenic C3 plant expressing a heterologous protein according to the SEQ ID NO. 1, SEQ ID NO. 2-3, and/or a heterologous protein having 73% homology with SEQ ID NOs. 1 and/or 2-3, as described herein which exhibits enhanced CO₂ fixation and/or CO₂ fixation compared to an otherwise identical control plant grown under the same conditions, for example wherein CO₂ fixation may be enhanced in the range of from about 10% to about 50% compared to that of an otherwise identical control plant grown under the same conditions.

Another embodiment of the present invention provides for a transgenic C3 plant expressing a heterologous protein according to the SEQ ID NO. 1, SEQ ID NO. 2-3, and/or a heterologous C4 photosynthetic Fd or Fd protein having 73% homology with SEQ ID NOs. 1 and/or 2-3, as described herein which exhibits enhanced photosynthetic electron transfer rates compared to an otherwise identical control plant grown under the same conditions, for example, wherein enhanced photosynthetic electron transfer rates may be enhanced in the range of from about 10% to about 50% compared to that of an otherwise identical control plant grown under the same conditions.

Another embodiment of the present invention provides for a transgenic C3 plant expressing a heterologous protein according to the SEQ ID NO. 1, SEQ ID NO. 1, and/or a heterologous C4 photosynthetic Fd or Fd protein having 73% homology with SEQ ID NOs. 1 and 2, as described herein which exhibits enhanced biomass accumulation compared to an otherwise identical control plant grown under the same conditions, for example wherein biomass accumulation may be enhanced in the range of from about 1- to 3-fold compared to that of an otherwise identical control plant grown under the same conditions.

Another embodiment of the present invention provides for a transgenic C3 plant expressing a heterologous protein according to the SEQ ID NO. 1, SEQ ID NO. 2-3, and/or a heterologous C4 photosynthetic Fd or Fd protein having 73% homology with SEQ ID NOs. 1 and/or 2-3, as described herein which exhibits enhanced abiotic tolerance compared to an otherwise identical control plant grown under the same conditions. For example, in a select transgenic strain, cold and heat stress tolerance may be enhanced in of the photosynthetic apparatus including the level of non-photochemical quenching (NPQ) as well as accelerating its rate of decay in the dark increasing the efficiency of photon utilization for photosynthesis, elevated levels of open photosystem II complexes, and increased linear electron transfer rates following stress application compared to wild type plants grown under the same conditions.

Another embodiment provides for a part of said transgenic plant of any embodiment described herein. For example, the part of said transgenic plant may be selected from among a protoplast, a cell, a tissue, an organ, a cutting, an explant, a reproductive tissue, a vegetative tissue, biomass, an inflorescence, a flower, a sepal, a petal, a pistil, a stigma, a style, an ovary, an ovule, an embryo, a receptacle, a seed, a fruit, a stamen, a filament, an anther, a male or female gametophyte, a pollen grain, a meristem, a terminal bud, an axillary bud, a leaf, a stem, a root, a tuberous root, a rhizome, a tuber, a stolon, a corm, a bulb, an offset, a cell of said plant in culture, a tissue of said plant in culture, an organ of said plant in culture, a callus, propagation materials, germplasm, cuttings, divisions, and propagations.

Another embodiment provides for a progeny or derivative of said transgenic C3 plant expressing a C4 photosynthetic Fd of any embodiment described herein. For example, the progeny or derivatives may be selected from among clones, hybrids, samples, seeds, and harvested material thereof and may be produced sexually or asexually.

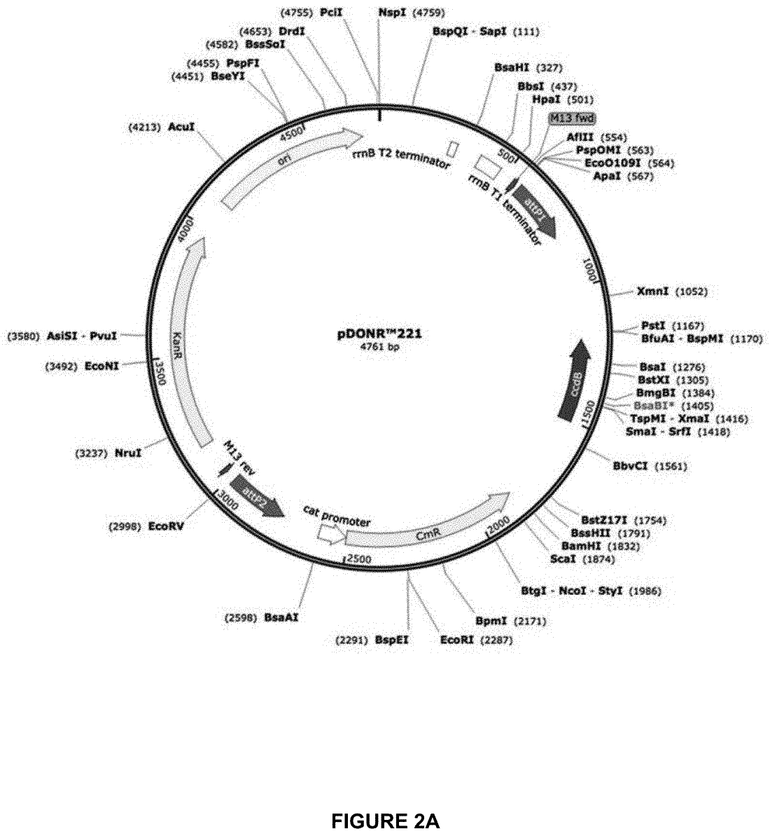

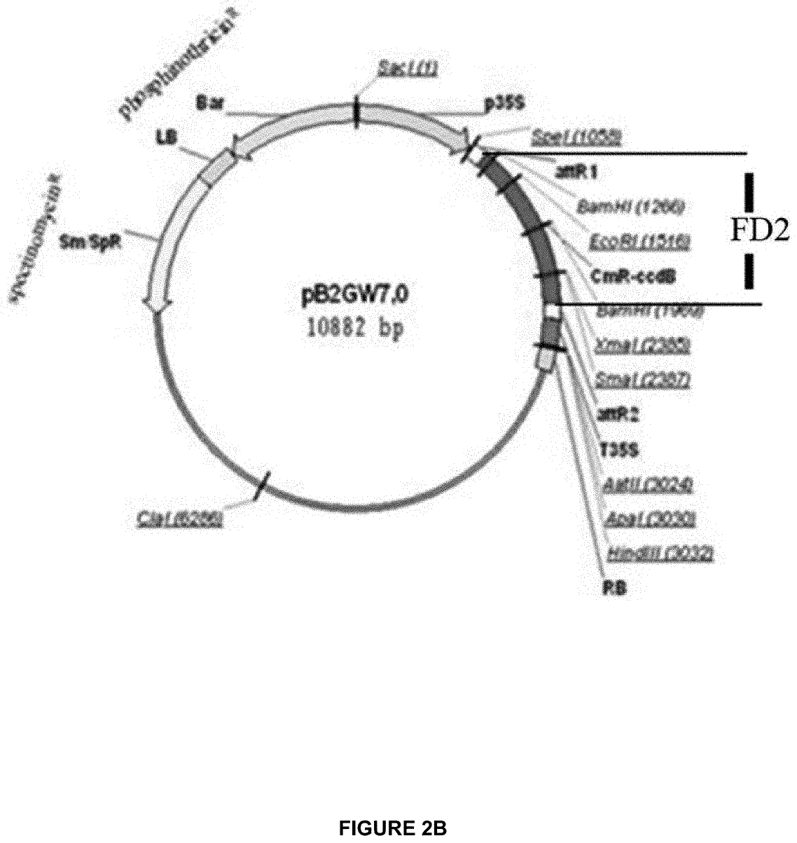

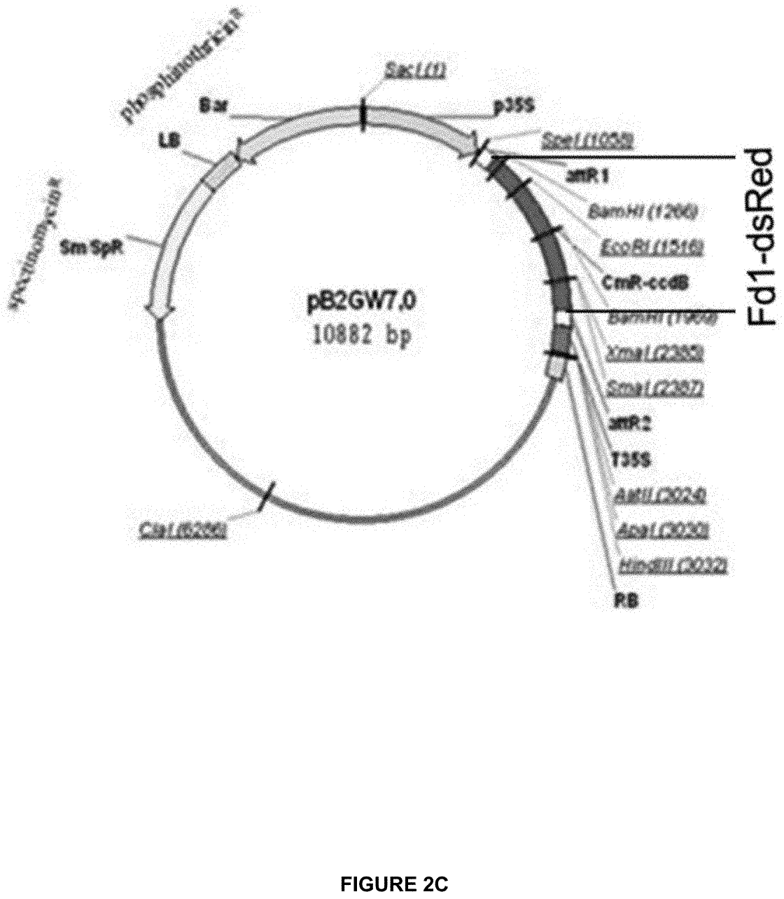

Another embodiment provides for use of a construct comprising one or more nucleic acids encoding a photosynthetic Fd2 protein, or a variant or homologue or homolog thereof. In one embodiment, this construct may include one or more nucleic acids encoding a heterologous Fd2 protein, or a variant or homologue thereof. In certain embodiment, the Fd2 gene may be operably linked to a promotor. In one preferred embodiment, this construct may include one or more nucleic acids encoding a heterologous Fd2 protein identified as SEQ ID NOs. 1, or a variant or homologue thereof, operably linked to a promotor. In a preferred embodiment, this construct may be identified in FIG. 2, wherein the photosynthetic Fd2 nucleotide sequence may be identified as SEQ ID No. 4 or 5, or a variant or homolog thereof, operably linked to a promotor.

Another embodiment provides for use of a construct comprising one or more nucleic acids encoding a photosynthetic Fd protein, or a variant or homologue thereof. In one embodiment, this construct may include one or more nucleic acids encoding a heterologous Fd protein, or a variant or homologue thereof. In certain embodiment, the Fd gene may be operably linked to a promotor. In one preferred embodiment, this construct may include one or more nucleic acids encoding a heterologous Fd2 protein identified as SEQ ID NOs. 2 or 3, or a variant or homologue thereof, operably linked to a promotor. In a preferred embodiment, this construct may be identified in FIG. 2, wherein the photosynthetic Fd1 nucleotide sequence may be identified as SEQ ID No. 6 or 7, or a variant or homolog thereof, operably linked to a promotor.

Another embodiment provides for use of a construct comprising one or more nucleic acids encoding a photosynthetic Fd protein, or a variant or homologue thereof. In one embodiment, this construct may include one or more nucleic acids encoding a heterologous Fd protein, or a variant or homologue thereof. In certain embodiment, the Fd gene may be operably linked to a promotor. In one preferred embodiment, this construct may include one or more nucleic acids encoding a heterologous Fd2 protein identified as SEQ ID NOs. 8, 9, or 10, or a variant or homologue thereof, operably linked to a promotor. In a preferred embodiment, this construct may be according to FIG. 2, wherein the photosynthetic Fd nucleotide sequence, or a variant or homolog thereof, may be operably linked to a promotor.

In certain preferred embodiment, C4 ferredoxin sequences, and preferable Fd1 and/or Fd2 sequences, can be identified from any C4 photosynthetic organism. For example, certain C4 Fd1 or Fd2 sequences such as nucleotide sequences according to SEQ ID NOs. 4-7 can be isolated from Zea mays. C4 Fd1 and Fd2 amino acid sequences, such as SEQ ID NOs. 1-3, can be isolated from Zea mays. Additionally, orthologs of C4 Fd sequences can also be identified in different photosynthetic organisms. “Orthologs” is intended to mean genes derived from a common ancestral gene and which are found in different species as a result of speciation. Genes found in different species are considered orthologs when their nucleotide sequences and/or their encoded protein sequences share at least about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. As shown in the figures below, a variety of coding sequences having certain homologies may be employed within the invention. For example, the amino acid sequences Fd1 and Fd2 demonstrate a sequence identity or homology of 73%. In other example, the amino acid sequences of different Fd1 and Fd1 (SEQ ID NO. 2 and SEQ ID NO. 3) demonstrate a sequence identity or homology of 98%. Additional amino acid and peptide sequences identified herein may exhibit similar sequence identify rages which are specifically included in the inventive technology. In addition, such sequence identities between polynucleotide further accounts for gene sequences identified here, as well as polynucleotide sequences that encode a specific photosynthetic Fd mRNA which are provided below.

The C4 photosynthetic Fd sequences, and preferable Fd1 and/or Fd2 sequences, can be provided in DNA constructs or expression cassettes for expression of a C4 photosynthetic Fd in a C3 plant of interest. The expression cassette may include a promoter sequence active in a C3 plant cell operably linked to a C4 Fd sequence. The cassette may additionally contain at least one additional gene to be co-transformed into the organism in some embodiments. Multiple C4 photosynthetic Fd sequences, such as FD1 (SEQ ID NO. 6-7), and FD2 (SEQ ID NO. 4 or 5) can be provided on a single expression cassette under the control of a single promoter or on a single expression cassette under the control of multiple promoters. In addition, multiple C4 photosynthetic Fd sequences encoding the full gene, or mRNA to be translated into a specific Fd protein, such as an FD1 protein (SEQ ID NO. 2-3), and/or FD2 protein (SEQ ID NO. 1) can be provided on a single expression cassette under the control of a single promoter or on a single expression cassette under the control of multiple promoters, among other variations.

Alternatively, C4 photosynthetic Fd sequences can be provided on multiple expression cassettes. As generally shown in FIG. 2, such an expression cassette is provided with a plurality of restriction sites and/or recombination sites for insertion of the C4 photosynthetic Fd sequence to be under the transcriptional regulation of the operably linked promoter. The expression cassette may additionally contain selectable marker genes. In certain embodiments, polynucleotide sequences encoding C4 photosynthetic Fd that have similar functions are expressed together in a plant. For example, C4 Ferredoxin sequences expressed in conjunction with a 4-gene construct that enhances CO2 concentration in C3 plant chloroplasts as taught by Sayre et al., in U.S. patent application Ser. No. 15/411,854 having the following nucleotide sequences: HLA3, PGR5, LCIA, BCA. (Such gene and protein sequences and their methods of transformation and expression are hereby incorporated specifically by reference). Thus, polynucleotides encoding different C4 Ferredoxins can be provide on the same expression cassette or different expression cassettes. Likewise, polynucleotides encoding different C4 Ferredoxins can be operably linked to the same promoter or different promoters. Such exemplary expression cassettes may include in the 5′-3′ direction of transcription, a transcriptional and translational initiation region (i.e., a promoter), a polynucleotide encoding at least one C4 Ferredoxin protein, and a transcriptional and translational termination region (i.e., termination region) functional in C3 plants.

In addition, a “variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired C4 Ferredoxin biological activity of the native plant protein, and more preferably a FD1 and/or FD2 from a C4 plant. Biologically active variants of a native C4 Ferredoxin proteins disclosed herein will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a C4 Ferredoxin protein, such as Fd1 or Fd2, or Fd1 and Fd1 variants, or Fd2 and Fd2 variants, disclosed herein may differ from that protein by as few as 1-15 amino acid residues, as few as 1-10, such as 6-10, as few as 5, as few as 4, 3, 2, or even 1 amino acid residue. Biologically active variants of C4 Ferredoxins retain C4 Ferredoxin activity. As used herein, “C4 Ferredoxin activity” refers to the ability of the C4 Ferredoxin to function within the plant's photosynthetic system as generally described herein.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J Mol. Biol. 48:443-453; the search-for-local alignment method of Pearson and Lipman (1988) Proc. Natl. Acad. Sci. USA 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Variant sequences can be isolated by PCR. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

The C4 photosynthetic Fd sequences disclosed herein when assembled within a promoter such that the promoter is operably linked to a nucleotide sequence encoding a C4 Fd protein, enable expression of the C4 Fd sequence in the cells of a plant stably or transiently transformed with this DNA construct.

The following definitions are provided to aid the reader in understanding the various aspects of the present disclosure. Unless defined otherwise, all technical and scientific terms used herein have the same meaning as commonly understood by those of ordinary skill in the art to which the disclosure pertains.

As used herein and in the appended claims, the singular forms “a”, “an”, and “the” include plural referents unless the context clearly dictates otherwise. Thus, for example, reference to “a plant” includes a plurality of such plants; reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The term “comprising” as used in a claim herein is open-ended, and means that the claim must have all the features specifically recited therein, but that there is no bar on additional features that are not recited being present as well. The term “comprising” leaves the claim open for the inclusion of unspecified ingredients even in major amounts. The term “consisting essentially of” in a claim means that the invention necessarily includes the listed ingredients, and is open to unlisted ingredients that do not materially affect the basic and novel properties of the invention. A “consisting essentially of” claim occupies a middle ground between closed claims that are written in a closed “consisting of” format and fully open claims that are drafted in a “comprising′ format”. These terms can be used interchangeably herein if, and when, this may become necessary. Furthermore, the use of the term “including”, as well as other related forms, such as “includes” and “included”, is not limiting.

As used herein a “wild type” or “wild type plant” or “control plant” means a plant that does not contain the recombinant DNA that expressed a protein or element that imparts an enhanced trait. A wild type, or control plant is to identify and select a transgenic plant that has an enhanced trait. A suitable wild type or control plant can be a non-transgenic plant of the parental line used to generate a transgenic organism, i.e. devoid of recombinant DNA. A “control plant” may include (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant a subject plant or plant cell, which may include progeny of a hemizygous transgenic plant that does not contain the recombinant DNA; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

Regarding disclosed ranges, the endpoints of all ranges directed to the same component or property are inclusive and independently combinable (e.g., ranges of “about 25%, or, more, about 5% to about 20 wt. %,” is inclusive of the endpoints and all intermediate values of the ranges of “about 5% to about 25%,” etc.). Numeric ranges recited with the specification are inclusive of the numbers defining the range and include each integer within the defined range.

Notably, all peptides disclosed in specifically encompass peptide having conservative amino acid substitutions. As used herein, “conservative amino acid substitutions” means the manifestation that certain amino acids can be substituted for other amino acids in a protein structure without appreciable loss of biochemical or biological activity. Since it is the interactive capacity and nature of a protein that defines that protein's biological functional activity, certain amino acid sequence substitutions can be made in a protein sequence, and, of course, the underlying DNA coding sequence, and nevertheless obtain a protein with like properties. Thus, various changes can be made in the amino acid sequences disclosed herein, or in the corresponding DNA sequences that encode these amino acid sequences, without appreciable loss of their biological utility or activity.

Examples of amino acid groups defined in this manner include: a “charged polar group,” consisting of glutamic acid (Glu), aspartic acid (Asp), asparagine (Asn), glutamine (Gln), lysine (Lys), arginine (Arg) and histidine (His); an “aromatic, or cyclic group,” consisting of proline (Pro), phenylalanine (Phe), tyrosine (Tyr) and tryptophan (Trp); and an “aliphatic group” consisting of glycine (Gly), alanine (Ala), valine (Val), leucine (Leu), isoleucine (Ile), methionine (Met), serine (Ser), threonine (Thr) and cysteine (Cys).

Within each group, subgroups can also be identified, for example, the group of charged polar amino acids can be sub-divided into the sub-groups consisting of the “positively-charged sub-group,” consisting of Lys, Arg and His; the negatively-charged sub-group,” consisting of Glu and Asp, and the “polar sub-group” consisting of Asn and Gin. The aromatic or cyclic group can be sub-divided into the sub-groups consisting of the “nitrogen ring sub-group,” consisting of Pro, His and Trp; and the “phenyl sub-group” consisting of Phe and Tyr. The aliphatic group can be sub-divided into the sub-groups consisting of the “large aliphatic non-polar sub-group,” consisting of Val, Leu and Ile; the “aliphatic slightly-polar sub-group,” consisting of Met, Ser, Thr and Cys; and the “small-residue sub-group,” consisting of Gly and Ala.

Examples of conservative mutations include substitutions of amino acids within the sub-groups above, for example, Lys for Arg and vice versa such that a positive charge can be maintained; Glu for Asp and vice versa such that a negative charge can be maintained; Ser for Thr such that a free —OH can be maintained; and Gin for Asn such that a free —NH2 can be maintained.

Proteins and peptides biologically functionally equivalent to the proteins and peptides disclosed herein include amino acid sequences containing conservative amino acid changes in the fundamental amino acid sequence. In such amino acid sequences, one or more amino acids in the fundamental sequence can be substituted, for example, with another amino acid(s), the charge and polarity of which is similar to that of the native amino acid, i.e., a conservative amino acid substitution, resulting in a silent change. It should be noted that there are a number of different classification systems in the art that have been developed to describe the interchangeability of amino acids for one another within peptides, polypeptides, and proteins. The following discussion is merely illustrative of some of these systems, and the present disclosure encompasses any of the “conservative” amino acid changes that would be apparent to one of ordinary skill in the art of peptide, polypeptide, and protein chemistry from any of these different systems. Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 1a, infra, contains information about which nucleic acid codons encode which amino acids.

TABLE 1a
Amino acid Nucleic acid codons

| Amino Acid | Nucleic Acid Codons |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |

“Control” or “control level” means the level of a molecule, such as a polypeptide or nucleic acid, normally found in nature under a certain condition and/or in a specific genetic background. In certain embodiments, a control level of a molecule can be measured in a cell or specimen that has not been subjected, either directly or indirectly, to a treatment. A control level is also referred to as a wildtype or a basal level. These terms are understood by those of ordinary skill in the art. A control plant, i.e. a plant that does not contain a recombinant DNA that confers (for instance) an enhanced trait in a transgenic plant, is used as a baseline for comparison to identify an enhanced trait in the transgenic plant. A suitable control plant may be a non-transgenic plant of the parental line used to generate a transgenic plant. A control plant may in some cases be a transgenic plant line that comprises an empty vector or marker gene, but does not contain the recombinant DNA, or does not contain all of the recombinant DNAs, in the test plant.

The term “enhanced” may refer to an enhanced trait, or phenotype which as used herein refers to a measurable improvement in a trait of plant or plant cell including, but not limited to, photosynthesis, photosynthetic electron transfer, carbon fixation rates, yield increase, including increased yield under non-stress conditions and increased yield under environmental stress conditions, biomass increases, above-ground biomass increases, increases abiotic stress tolerance. “abiotic stress” as used herein includes drought (water deficit), excessive watering (water-logging/flooding), extreme temperatures (cold, frost and heat), salinity (sodicity) and mineral (metal and metalloid) toxicity negatively impact growth, development, yield and seed quality of crop and other plants or plant cells.

By “yield” or “crop yield” is intended the measurement of the amount of a crop that was harvested per unit of land area. Crop yield is the measurement often used for grains or cereals and is typically measured as the amount of plant harvested per unit area for a given time, i.e., metric tons per hectare or kilograms per hectare. Crop yield can also refer to the actual seed or biomass produced or generated by the plant. In specific embodiments, expressing one or more C4 Fd protein, such as FD1, FD2 or both FD1 and FD2, in a C3 plant can increase the yield of the plant by at least 1%, 2%, 3%, 4%, 5%, 6%, 7%, 8%, 9%, 10%, 12%, 15%, 20%, 25%, 30%, 40%, 50%, 60%, 70%, 80%, 90%, 100%, or more when compared to the same plant without an expression of a heterologous Fd protein. Methods to measure yield are commonly known in the art. Yield may refer to yields of specific plant products, such as products selected from among starches, oils, fatty acids, triacylglycerols, lipids, cellulose or other carbohydrates, alcohols, sugars, nutraceuticals, pharmaceuticals, fragrance and flavoring compounds, and organic acids.

Moreover, the terms “enhance”, “enhanced”, “increase”, or “increased” refer to a statistically significant increase, for example in a plant trait or phenotype. For the avoidance of doubt, these terms generally refer to about a 5% increase in a given parameter or value, about a 10% increase, about a 15% increase, about a 20% increase, about a 25% increase, about a 30% increase, about a 35% increase, about a 40% increase, about a 45% increase, about a 50% increase, about a 55% increase, about a 60% increase, about a 65% increase, about 70% increase, about a 75% increase, about an 80% increase, about an 85% increase, about a 90% increase, about a 95% increase, about a 100% increase, or more over the control value. These terms also encompass ranges consisting of any lower indicated value to any higher indicated value, for example “from about 5% to about 50%”, etc.

“Expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g., intrans) or may lack such intervening_non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression cassette or “expression vector” or “vector” refers to a nucleic acid construct, which when introduced into a host cell, results in transcription and/or translation of a RNA or polypeptide, respectively.

The term “genome” as it applies to a plant cells encompasses not only chromosomal DNA found within the nucleus, but organelle DNA found within subcellular components (e.g., mitochondrial, plastid) of the cell. As used herein, the term “genome” refers to the nuclear genome unless indicated otherwise. However, expression in a plastid genome, e.g., a chloroplast genome, or targeting to a plastid genome such as a chloroplast via the use of a plastid targeting sequence, is also encompassed by the present disclosure.

The term “heterologous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. Thus, the organism can have more than the usual number of copy(ies) of such fragment located in its (their) normal position within the genome and in addition, in the case of plant cells, within different genomes within a cell, for example in the nuclear genome and within a plastid or mitochondrial genome as well. A nucleic acid fragment that is heterologous with respect to an organism into which it has been inserted or transferred is sometimes referred to as a “transgene.”

A “heterologous” FD1 or FD2 protein or FD1 or Fd2 protein-encoding nucleotide sequence, etc., can be one or more additional copies of an endogenous FD2 protein or Fd2 protein-encoding nucleotide sequence, or a nucleotide sequence from another plant or other source. Furthermore, these can be genomic or non-genomic nucleotide sequences. Non-genomic nucleotide sequences encoding such proteins and peptides include, by way of non-limiting examples, mRNA; synthetically produced DNA including, for example, cDNA and codon-optimized sequences for efficient expression in different transgenic plants reflecting the pattern of codon usage in such plants; nucleotide sequences encoding the same proteins or peptides, but which are degenerate in accordance with the degeneracy of the genetic code; which contain conservative amino acid substitutions that do not adversely affect their activity, etc., as known by those of ordinary skill in the art.

The term “homology” describes a mathematically based comparison of sequence similarities which is used to identify genes or proteins with similar functions or motifs. The nucleic acid and protein sequences of the present invention can be used as a “query sequence” to perform a search against public databases to, for example, identify other family members, related sequences, or homologs. The term “homologous” refers to the relationship between two nucleic acid sequence and/or proteins that possess a “common evolutionary origin”, including nucleic acids and/or proteins from superfamilies (e.g., the immunoglobulin superfamily) in the same species of animal, as well as homologous nucleic acids and/or proteins from different species of animal (for example, myosin light chain polypeptide, etc.; see Reeck et al., (1987) Cell, 50:667). Such proteins (and their encoding nucleic acids) may have sequence homology, as reflected by sequence similarity, whether in terms of percent identity or by the presence of specific residues or motifs and conserved positions. The methods disclosed herein contemplate the use of the presently disclosed nucleic and protein sequences, as well as sequences having sequence identity and/or similarity, and similar function.

“Host cell” means a cell which contains an expression vector and supports the replication and/or expression of that vector. The term “introduced” means providing a nucleic acid (e.g., an expression construct) or protein into a cell. “Introduced” includes reference to the incorporation of a nucleic acid into a eukaryotic or prokaryotic cell where the nucleic acid may be incorporated into the genome of the cell, and includes reference to the transient provision of a nucleic acid or protein to the cell. “Introduced” includes reference to stable or transient transformation methods, as well as sexually crossing. Thus, “introduced” in the context of inserting a nucleic acid fragment (e.g., a recombinant DNA construct/expression construct) into a cell, can mean “transfection” or “transformation” or “transduction”, and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

As used herein, “nucleic acid” or “nucleotide sequence” means a polynucleotide (or oligonucleotide), including single or double-stranded polymers of deoxyribonucleotides or ribonucleotide bases, and unless otherwise indicated, encompasses naturally occurring and synthetic nucleotide analogues having the essential nature of natural nucleotides in that they hybridize to complementary single stranded nucleic acids in a manner similar to naturally occurring nucleotides. Nucleic acids may also include fragments and modified nucleotide sequences. Nucleic acids disclosed herein can either be naturally occurring, for example genomic nucleic acids, or isolated, purified, nongenomic nucleic acids, including synthetically produced nucleic acid sequences such as those made by solid phase chemical oligonucleotide synthesis, enzymatic synthesis, or by recombinant methods, including for example, cDNA, codon-optimized sequences for efficient expression in different transgenic plants reflecting the pattern of codon usage in such plants, nucleotide sequences that differ from the nucleotide sequences disclosed herein due to the degeneracy of the genetic code but that still encode the protein(s) of interest disclosed herein, nucleotide sequences encoding the presently disclosed protein(s) comprising conservative (or non-conservative) amino acid substitutions that do not adversely affect their normal activity, PCR-amplified nucleotide sequences, and other non-genomic forms of nucleotide sequences familiar to those of ordinary skill in the art.

“Nucleic acid construct” or “construct” refers to an isolated polynucleotide which can be introduced into a host cell. This construct may comprise any combination of deoxyribonucleotides, ribonucleotides, and/or modified nucleotides. This construct may comprise an expression cassette that can be introduced into and expressed in a host cell.

“Operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The terms “peptide”, “polypeptide”, and “protein” are used to refer to polymers of amino acid residues. These terms are specifically intended to cover naturally occurring biomolecules, as well as those that are recombinantly or synthetically produced, for example by solid phase synthesis.

The term “promoter” or “regulatory element” refers to a region or nucleic acid sequence located upstream or downstream from the start of transcription and which is involved in recognition and binding of RNA polymerase and/or other proteins to initiate transcription of RNA. Promoters need not be of plant or algal origin. For example, promoters derived from plant viruses, such as the CaMV35S promoter, or from other organisms, can be used in variations of the embodiments discussed herein. Promoters useful in the present methods include, for example, constitutive, strong, weak, tissue-specific, cell-type specific, seed-specific, inducible, repressible, and developmentally regulated promoters.

Notably, as shown in FIG. 2, and incorporated herein, a large number of promoters, including constitutive, inducible and repressible promoters, from a variety of different sources are well known in the art. Representative sources include for example, algal, viral, mammalian, insect, plant, yeast, and bacterial cell types, and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters active in plants include, for example nopaline synthase (nos) promoter and octopine synthase (ocs) promoters carried on tumor-inducing plasmids of Agrobacterium tumefaciens and the method wherein the tissue targeting sequence is chosen from sequences promoters such as the Cauliflower Mosaic Virus (CaMV) 19S or 35S promoter (U.S. Pat. No. 5,352,605), CaMV 35S promoter with a duplicated enhancer (U.S. Pat. Nos. 5,164,316; 5,196,525; 5,322,938; 5,359,142; and 5,424,200), and the Figwort Mosaic Virus (FMV) 35S promoter (U.S. Pat. No. 5,378,619). These promoters and numerous others have been used in the creation of constructs for transgene expression in plants or plant cells. Other useful promoters are described, for example, in U.S. Pat. Nos. 5,391,725; 5,428,147; 5,447,858; 5,608,144; 5,614,399; 5,633,441; 6,232,526; and 5,633,435, all of which are incorporated herein by reference. Additional useful light inducible promoters include but not limited to are: (1) PPCZm1 (phosphoenolpyruvate carboxylase from corn) Kausch et al. (2001) Plant Molecular Biology 45, 1-15; (2) RbcS (ribulose-bisphosphate carboxylase from rice) Nomura et al. (2000) The Plant Journal 22(3), 211-221 (3) Rca (Rubisco Activase from rice) Yang et al. (2012) Biochemical and Biophysical Research Communications 418, 565-570 (4) LHCP2 (light harvesting chlorophyll a/b binding-protein from rice) Tada et al. (1991), EMBO J. 10(7), 1803-1808 (5) cyFBPase (cytosolic fructose 1,6 biphosphatase from rice) Si et al., 2002, Acta Botanica Sinica. 44(11), 1339-1345. In some embodiments the promoter will be a light-inducible promoter such as the promoter for rbcS, CAB1, Dofl, psbD, PPDK, PPCZm1, Rca, LHCP2, cyFBPase and the like.

Alteration of a C4 Ferredoxin gene expression may also be achieved through the modification of DNA in a way that does not alter the sequence of the DNA. Such changes could include modifying the chromatin content or structure of the C4 Ferredoxin gene of interest and/or of the DNA surrounding the C4 Ferredoxin gene. It is well known that such changes in chromatin content or structure can affect gene transcription (Hirschhorn et al. (1992) Genes and Dev 6:2288-2298; Narlikar et al. (2002) Cell 108: 475-487). Such changes could also include altering the methylation status of the C4 Ferredoxin gene of interest and/or of the DNA surrounding the C4 Ferredoxin gene. It is well known that such changes in DNA methylation can alter transcription (Hsieh (1994) Mol Cell Biol 14: 5487-5494). It can be obvious to those skilled in the art that other similar alterations (collectively termed “epigenetic alterations”) to the DNA that regulates transcription of one or more C4 Ferredoxin genes of interest may be applied in order to achieve the desired result of an altered C4 Ferredoxin gene expression profile.

Alteration of C4 transporter gene expression may also be achieved through the use of transposable element technologies to alter gene expression. It is well understood that transposable elements can alter the expression of nearby DNA (McGinnis et al. (1983) Cell 34:75-84). Alteration of the expression of a gene encoding a C4 Ferredoxin in a photosynthetic organism may be achieved by inserting a transposable element upstream of the C4 Ferredoxin gene of interest, causing the expression of said gene to be altered.

As used herein, the phrase “identity” or “sequence identity” or “sequence similarity” is the similarity between two (or more) nucleic acid sequences, or two (or more) amino acid sequences. However, in common usage and in the instant application, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 85%, and more preferably at least about 90% or at least about 95% of the nucleotides match over a defined length of the nucleic acid sequences, as determined by a sequence comparison algorithm known such as BLAST, FASTA, DNA Strider, CLUSTAL, etc. An example of such a sequence is an allelic or species variant of the specific genes of the present invention. Sequences that are substantially homologous may also be identified by hybridization, e.g., in a Southern hybridization experiment under, e.g., stringent conditions as defined for that particular system.

Similarly, in particular embodiments of the invention, two amino acid sequences are “substantially homologous” or “substantially similar” when greater than 90% of the amino acid residues are identical. Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. The program may use the local homology algorithm of Smith and Waterman with the default values: Gap creation penalty=−(1+Ilk), k being the gap extension number, Average match=1, Average mismatch=—0.333.

Sequence identity is frequently measured as the percent of identical nucleotide or amino acid residues at corresponding positions in two or more sequences when the sequences are aligned to maximize sequence matching, i.e., taking into account gaps and insertions. The constructs and methods disclosed herein encompass nucleic acid and protein sequences, namely amino acid sequences according to SEQ ID NO. 1-3, and 8-10 and polynucleotide sequences according to SEQ ID NO. 4-7 respectively, having sequence identity/sequence similarity at least about 70%, 75%, 80%, 85%, 90%, 91%, 92%, 93%, 94%, 95%, 96%, 97%, 98%, 99%, 99.9%, or up to a single point mutation, to those specifically and/or sequences having the same or similar function for example if a protein or nucleic acid is identified with a transit peptide and the transit peptide is cleaved leaving the protein sequence without the transit peptide then the sequence identity/sequence similarity is compared to the protein with and/or without the transit peptide. Variants and homolog identified herein are generally considered to be include all sequences having “sequence identity” or “sequence similarity.” Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs.

One example of an algorithm that is suitable for determining percent sequence identity and sequence similarity is the BLAST algorithm, which is described in (Altschul, S., et al., NCBI NLM NIH Bethesda, Md. 20894; & Altschul, S., et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold.

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always; 0) and N (penalty score for mismatching residues; always; 0). For amino acid sequences, a scoring matrix is used to calculate the cumulative score. Extension of the word hits in each direction are halted when the −27 cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

A “transgenic” or “transformed” or “genetically modified” organism, such as a transgenic plant or cell, is a host organism that has been stably or transiently genetically engineered to contain one or more heterologous nucleic acid fragments, including nucleotide coding sequences, expression cassettes, vectors, etc. Introduction of heterologous nucleic acids into a host cell to create a transgenic cell is not limited to any particular mode of delivery, and includes, for example, microinjection, floral dip, adsorption, electroporation, vacuum infiltration, particle gun bombardment, whiskers-mediated transformation, liposome-mediated delivery, the use of viral and retroviral vectors, etc., as is well known to those skilled in the art.

As used herein, a “genetically modified plant or “transgenic plant” is one whose genome has been altered by the incorporation of exogenous genetic material, e.g. by transformation as described herein. The term “transgenic plant” is used to refer to the plant produced from an original transformation event, or progeny from later generations or crosses of a transgenic plant so long as the progeny contains the exogenous genetic material in its genome. By “exogenous” is meant that a nucleic acid molecule, for example, a recombinant DNA, originates from outside the plant into which it is introduced. An exogenous nucleic acid molecule may comprise naturally or non-naturally occurring DNA, and may be derived from the same or a different plant species than that into which it is introduced.

The C4 ferrodoxin genes disclosed herein can be used in expression cassettes to transform plants of interest. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320 334), electroporation (Riggs et al. (1986) Proc. Natl. Acad. Sci. USA 83:5602 5606, Agrobacterium-mediated transformation (U.S. Pat. Nos. 5,563,055 and 5,981,840), direct gene transfer (Paszkowski et al. (1984) EMBO J. 3:2717 2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923 926); and Lec1 transformation (WO 00/28058). Also see Weissinger et al. (1988) Ann. Rev. Genet. 22:421 477; Sanford et al. (1987) Particulate Science and Technology 5:27 37 (onion); Christou et al. (1988) Plant Physiol. 87:671 674 (soybean); McCabe et al. (1988) Bio/Technology 6:923 926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736 740 (rice); Klein et al. (1988) Proc. Natl. Acad. Sci. USA 85:4305 4309 (maize); Klein et al. (1988) Biotechnology 6:559 563 (maize); U.S. Pat. Nos. 5,240,855; 5,322,783; and, 5,324,646; Klein et al. (1988) Plant Physiol. 91:440 444 (maize); Fromm et al. (1990) Biotechnology 8:833 839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional methods. See, for example, McCormick et al. (1986) Plant Cell Reports 5:81-84. In this manner, the present invention provides transformed seed (also referred to as “transgenic seed”) having a polynucleotide of the invention, for example, an expression cassettes disclosed herein, stably incorporated into their genome.

One exemplary transformation method includes employing Agrobacterium tumefaciens or Agrobacterium rhizogenes as the transforming agent to transfer heterologous DNA into the plant. In this general embodiment, Typically, a plant cell, an explant, a meristem or a seed is infected with Agrobacterium tumefaciens transformed with the expression vector/construct which contains the heterologous nucleic acid operably linked to a promoter. Under appropriate conditions known in the art, the transformed plant cells are grown to form shoots, roots, and develop further into genetically altered plants. In some embodiments, the heterologous nucleic acid can be introduced into plant cells, by means of the Ti plasmid of Agrobacterium tumefaciens. The Ti plasmid is transmitted to plant cells upon infection by Agrobacterium tumefaciens, and is stably integrated into the plant genome.

“Stable transformation” is intended to mean that the nucleotide construct introduced into a plant integrates into the genome of the plant and is capable of being inherited by the progeny thereof. The nucleic acid molecule can be transiently expressed or non-stably maintained in a functional form in the cell for less than three months i.e. is transiently expressed.

The terms “plant” or “plants” that can be used in the present methods broadly include the classes of higher and lower plants amenable to transformation techniques, including angiosperms (monocotyledonous and dicotyledonous plants), gymnosperms, ferns, and unicellular and multicellular algae. The term “plant” also includes plants which have been modified by breeding, mutagenesis, or genetic engineering (transgenic and non-transgenic plants). It includes plants of a variety of ploidy levels, including aneuploid, polyploid, diploid, haploid, and hemizygous. The plant may be in any form including suspension cultures, embryos, meristematic regions, callus tissue, gametophytes, sporophytes, pollen, microspores, whole plants, shoot vegetative organs/structures (e.g. leaves, stems and tubers), roots, flowers and floral organs/structures, seed (including embryo, endosperm, and seed coat) and fruit, plant tissue (e.g. vascular tissue, ground tissue, and the like) and cells, and progeny of same.

While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The invention encompasses isolated or substantially purified C4 Ferredoxin polynucleotides or amino acid compositions. An “isolated” or “purified” C4 Ferredoxin polynucleotide or protein, or biologically active portion thereof, is substantially or essentially free from components that normally accompany or interact with the C4 Ferredoxin polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived.

A “variant,” or “isoform,” or “protein variant” is a member of a set of similar proteins that perform the same or similar biological roles. For example, fragments and variants of the disclosed C4 Ferredoxin polynucleotides and amino acid sequences encoded thereby are also encompassed by the present invention. By “fragment” is intended a portion of the polynucleotide or a portion of the amino acid sequence. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular C4 Ferredoxin disclosed herein will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Moreover, while polynucleotide sequences may be presented for one or more Ferredoxins, such sequences may represent pre-processed sequences in some instances which may contain un-excised portions. As such, the invention, when referring any polynucleotide sequences specifically references that sequences and/or the processed sequences, or variants, that directly codes for the subject protein.

The compositions disclosed herein also comprise synthetic oligonucleotides or nucleotide sequences encoding C4 Fd sequences. A synthetic sequence is one that is produced or reproduced in a laboratory setting. While the nucleotide sequence may have an altered nucleotide sequence relative to the parent sequence, the synthetic sequence may be identical to the naturally occurring sequence. In both instances, however, the structure of the synthetic sequence is altered or different from that found in the sequence that is directly isolated from its natural setting.

The term “C3 plant(s)” refers to plants which fix CO₂ in a C3 pathway of photosynthesis. The term “C4 plant(s)” refers to plants which fix CO₂ in a C4 pathway of photosynthesis.

The term “photosynthetic ferredoxin” or “ferredoxin” or “FD” of “Fd’ as used herein includes all naturally-occurring and synthetic forms of ferredoxin, whether bundle sheath cell specific and/or mesophyll cell specific that retain their specific activity in photosynthesis. Such ferredoxin proteins include the ferredoxin proteins include the protein from C4 plants, such as Maize (Zea Mays), as well as peptides derived from other C4 plant species and genera. The term “ferredoxin” or “FD” also encompasses one or more nucleotide sequences that encode a peptide that exhibits the function of a ferredoxin peptide. The term “ferredoxin” or “ferredoxin family proteins” include both ferredoxin and ferredoxin-like proteins in which the ferredoxin-like protein has sequence similarity to ferredoxin and contains a 2Fe-2S iron-sulfur cluster binding domain. The ferredoxin family proteins are electron carrier proteins with an iron-sulfur cofactor that act in a wide variety of metabolic reactions. A protein with electron carrier activity is a protein that serves as an electron acceptor and electron donor in an electron transport system. Ferredoxins can be divided into several subgroups depending upon the physiological nature of the iron-sulfur cluster(s) and according to sequence similarities.

The term “ferredoxin-1” or “FD1” as used herein includes all naturally-occurring and synthetic forms of ferredoxin-1 that retain it specific activity. Such ferredoxin-1 proteins include the protein from C4 plants, such as Maize (Zea Mays), as well as peptides derived from other C4 plant species and genera. The term “ferredoxin-1” or “FD1” also encompasses one or more nucleotide sequences that encode a peptide that exhibits the function of a ferredoxin-1 peptide.

The term “ferredoxin-2” or “FD2” as used herein includes all naturally-occurring and synthetic forms of ferredoxin-1 that retain it specific activity. Such ferredoxin-2 proteins include the protein from C4 plants, such as Maize (Zea Mays), as well as peptides derived from other C4 plant species and genera. The term “ferredoxin-2” or “FD2” also encompasses one or more nucleotide sequences that encode a peptide that exhibits the function of a ferredoxin-2 peptide.

Additionally, an “FD2 or FD2 protein,” or an “FD2 or FD1 protein from a C4 plant” any other protein or peptide presently broadly disclosed and utilized in any of the plants disclosed herein refers to a protein or peptide exhibiting enzymatic/functional activity similar or identical to the enzymatic/functional activity of the specifically named protein or peptide. Enzymatic/functional activities of the proteins and peptides disclosed herein are described below. “Similar” enzymatic/functional activity of a protein or peptide can be in the range of from about 75% to about 125% or more of the enzymatic/functional activity of the specifically named protein or peptide when equal amounts of both proteins or peptides are assayed, tested, or expressed as described below under identical conditions, and can therefore be satisfactorily substituted for the specifically named proteins or peptides in the present enhanced transgenic plants.

The terms “3C oilseed crop” or “3C oil crop” “oilseed plant/crop” or “oil plant/crop”, and the like, to which the present methods and compositions can also be applied, refer to C3 plants that produce seeds or fruit with oil content in the range of from about 1 to 2%, e.g., wheat, to about 20%, e.g., soybeans, to over 40%, e.g., sunflowers and rapeseed (canola). These include major and minor oil crops, as well as wild plant species which are used, or are being investigated and/or developed, as sources of biofuels due to their significant oil production and accumulation. Exemplary C3 oil seed crops or C3 oil crop plants useful in practicing the methods disclosed herein include, but are not limited to, plants of the genera Brassica (e.g., rapeseed/canola (Brassica napus; Brassica carinata; Brassica nigra; Brassica oleracea), Camelina, Miscanthus, and Jatropha; Jojoba (Simmondsia chinensis), coconut; cotton; peanut; rice; safflower; sesame; soybean; mustard; wheat; flax (linseed); sunflower; olive; corn; palm; palm kernel; sugarcane; castor bean; switchgrass; Baraga ojficinalis; Echium plantagineum; Cuphea hookeriana; Cuphea pulcherrima; Cuphea lanceolata; Ricinus communis; Coriandrum sativum; Crepis alpina; Vernonia galamensis; Momordica charantia; and Crambe abyssinica.

As used herein, a “3C food crop” or “food crop” means a C3 crop that has general commercial application, that may include human or animal consumption, or other commercial or industrial uses. Exemplary food crop plants include C3 crops wheat, rice, beans, barley, oats, sorghum, rye, and millet; peanuts, chickpeas, lentils, kidney beans, soybeans, lima beans; potatoes, sweet potatoes, and cassavas; soybeans, canola, peanuts, palm, coconuts, safflower, cottonseed, sunflower, flax, olive, and safflower; sugar cane and sugar beets; fruits, bananas, oranges, apples, pears, breadfruit, pineapples, and cherries; cucumbers, blueberries, raspberries, tomatoes, peppers, lettuce, carrots, melons, strawberry, asparagus, broccoli, peas, kale, cashews, peanuts, walnuts, pistachio nuts, almonds; forage and turf grasses; alfalfa, clover; coffee, cocoa, kola nut, poppy; vanilla, sage, thyme, anise, saffron, menthol, mint, Hops, stevia, calendula, vanilla, jasmine, basil, oregano, rosemary, cilantro, peppermint, watercress, wasabi, spearmint and coriander and preferably wheat, rice and canola.

Additional examples of C3 plants that may be within the inventive technology may include members of the family Cannabaceae, such as Cannabis, and hemp among others. Additional examples of C3 plants that may be within the inventive technology may algae that utilize C3 photosynthesis.

Exemplary C4 and C3 are readily identifiable by those of ordinary skill in the art. Exemplary C4 plants may be generally selected from the group consisting of genera Panicum, Saccharum, Setaria, Sorghum and Zea. Additional C4 plants may include, but not be limited to: corn, sorghum, sugarcane, millet, and switchgrass.

Additional exemplary C3 oil seed, oil crops, and food crops may be generally selected from the group consisting of: rice (Oryza sativa), wheat (Triticum spp.), barley (Hordeum vulgare), rye (Secale cereale), oat (Avena sativa); soybean (Gycine max), peanut (Arachis hypogaea), cotton (Gossypium spp.), sugar beets (Beta vulgaris), tobacco (Nicotiana tabacum), spinach (Spinacea oleracea), soybean (Glycine max), or potato (Solanum tuberosum), as well as petunia, tomato, carrot, cabbage, poplar, alfalfa, crucifers, Arabidopsis, and oilseed rape.

Additionally, as noted above: “CET” refers to cyclic electron transfer; “LET” refers to linear electron transfer; “WT” refers to wild-type; and “Fd” refers to ferredoxin.

Although the foregoing invention has been described in some detail by way of illustration and example for purposes of clarity of understanding, it will be obvious that certain changes and modifications may be practiced within the scope of the appended claims.

### EXAMPLES

### Example 1: Demonstrates Construction of Entry Clone and Expression Vectors Expressing the FD2 Gene Construct and Transformation Protocol

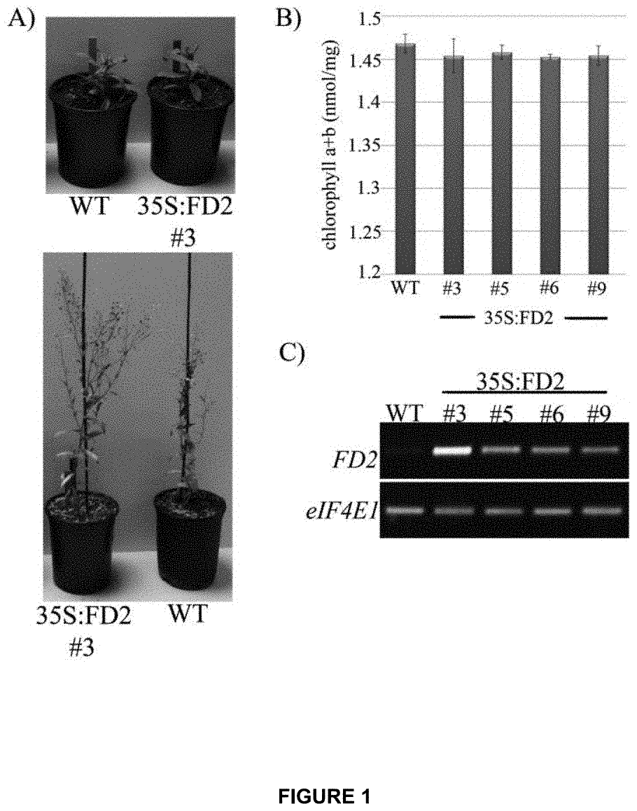

As demonstrated by the present inventors generated an exemplary plasmid cloning system as part of the invtive technology. As shown in FIG. 1, after performing a BP reaction, the gene of interest, in this instance FD2, may been inserted with attB recombination sites into a donor vector (pDONR221-KANR) containing attP sites to generate an entry clone. Naturally, expression cassettes, vectors and the like are exemplary only. Later, by performing an LR recombination reaction between an attL-containing entry clone and an attR-containing destination vector (pB2GW7-Spect.R) an expression clone or vector may be generated. In this embodiment, the exemplary pB2GW7 vector contains a 35S CaMV promoter. Each may be generally referred to as an expression cassette. In certain embodiments, one or more polynucleotides that encodes for one of the genes, and/or expression cassettes may be introduced to the plant using an expression vector for agrobacterium for the transformation.

The present inventors further demonstrated the transformation of Camelina sativa plants utilizing Agrobacterium-mediated transformation to deliver and expresses the heterologous polynucleotides as generally described herein. In this embodiment, Camelina plants were grown under a long-day light regime. The primary bolts were clipped when approximately 1-5 cm tall, and allowed to continue growing for approximately another week after clipping. Next, the present inventors prepared an Agrobacterium culture, having the polynucleotide of interest. In this embodiment, a 5 ml LB culture solution with a selective antibiotic added was prepared. In other embodiment, 1 ml of an overnight culture may be diluted into 1 L of fresh LB media and incubated for approximately 24 hours, under agitation conditions at about 28° C.

From this culture, the Agrobacterium cells may be concentrated and harvested. In this embodiment, the cell culture may be centrifuged at about 5K rpm for approximately 10 minutes and then resuspended in a standard infiltration medium. This infiltration medium may contain approximately: 1) half strength of MS salts (2.165 g/L); 2) 0.5 g/L MES; and 3) 50 g/L sucrose. Next, the present inventors removed the pods and fully-opened the Camelina flowers, and then added approximately, 200-500 ul of silwet L-77 to the infiltration solution forming an Agrobacterium solution, which was mixed well just before dipping. The present inventors then dipped above-ground part of the Camelina plants in the Agrobacterium solution for about 5 min with gentle shaking. Next, a plastic dome was placed over the dipped Camelina plants to maintain a high-level of humidity for approximately 24 hours, after which the plants were not watered for two days. Subsequent to this, the dipped Camelina plants were watered with appropriate quantities, after which dry seeds were harvested.

### Example 2: Characterization of 35S:FD2 Overexpressing Lines

Generally referring to FIGS. 2A-C, the present inventors have demonstrated and characterized the expression of four transgenic 35S:FD2 overexpressing lines. The present inventors demonstrated the phenotypic observation of an overexpressing CaMV 35S:FD2 line. In the young growth stage (25 days-old—not shown) an exemplary overexpressing plant or line shows no phenotypic difference from the WT. As shown in FIG. 2A, in a later growth stage (65 days-old) the overexpressing 35S:FD2 transgenic plant demonstrates a clear characteristic phenotype with more branching than the WT resulting in the production of more flowers and seed pods. As shown in FIG. 2B, the present inventors evaluated the chlorophyll content of leaves on a fresh weight basis. As demonstrated, the present inventors showed no significant difference in the chlorophyll levels of the four 35S:FD2 transgenic lines and the WT. As shown in FIG. 2C, the present inventors utilized reverse transcriptase-PCR (RT-PCR) to examine the overexpressing levels of the four 35S:FD2 transgenic lines using forward and reverse primers for the FD2gene. As demonstrated in the figure, Line 3 (#3) showed higher overexpressing levels comparing with the other three selected lines (#5, #6, and #9).

### Example 3: Characterization of Gas Exchange Measurements of 35S:FD2 High Overexpressing Lines Under Greenhouse Conditions

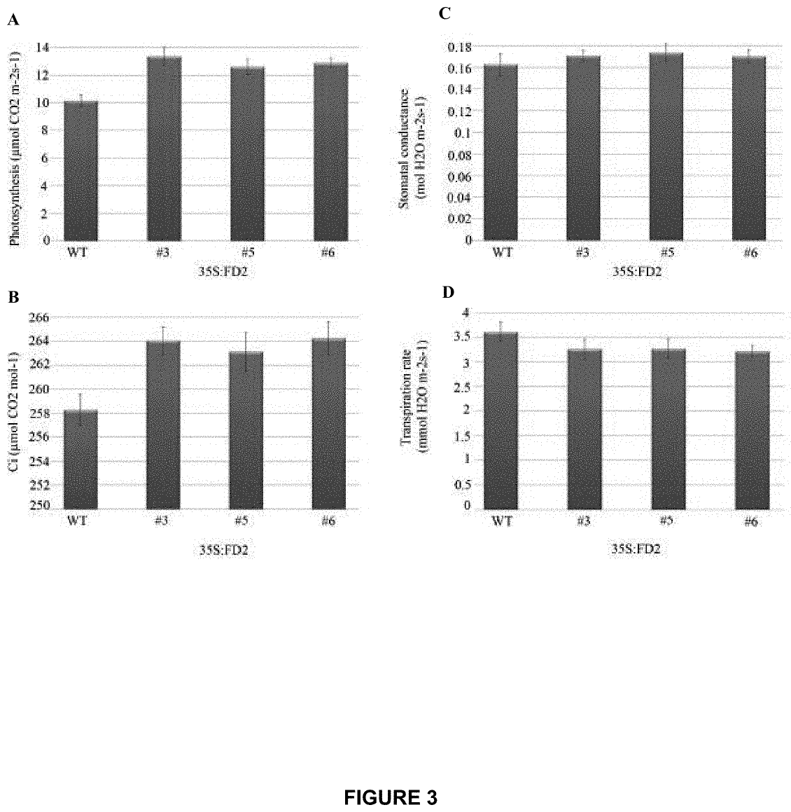

The present inventors performed gas exchange measurements under greenhouse conditions for three selected 35S:FD2 high overexpressing lines associated with substantially increased leaf internal CO₂ (Ci) levels. As generally shown in FIG. 3, at least three Fd2 lines show approximately a 25% higher photosynthetic rate and a 5-10% reduction in transpiration rates relative to WT.

### Example 4: Characterization of Gas Exchange Measurements of 35S:FD2 High Overexpressing Line Under Field Conditions

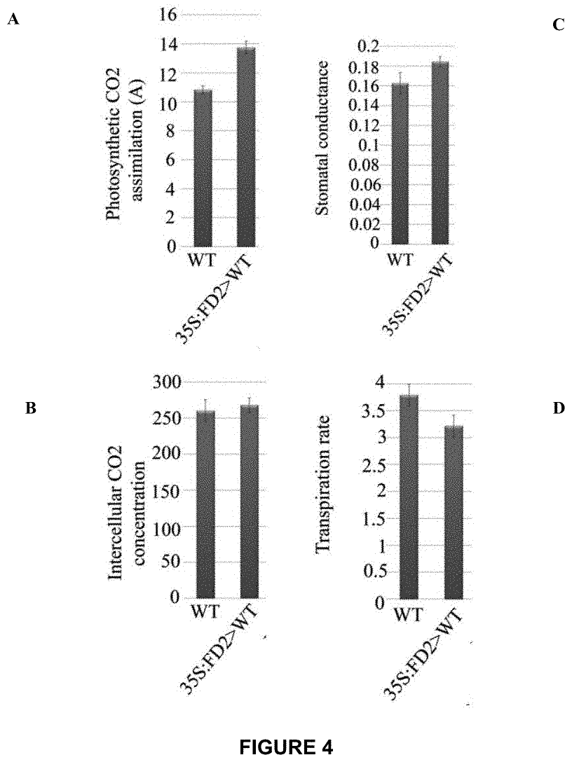

The present inventors performed gas exchange measurements under field conditions for a select 35S:FD2 high overexpressing line. As demonstrated in FIG. 4, gas exchange measurements revealed up to a 40% increase in CO₂ assimilation rate in 35S:FD2 overexpressing line comparing with the WT as well as a 30% decrease in the transpiration rate comparing with the WT. Intercellular CO₂ concentration was between 5-10% higher for the 35S:FD2 line comparing with the WT, while stomatal conductance levels were increased approximately 5% increase for the 35S:FD2 compared to WT.

### Example 5: Characterization of Plant Size and/or Seed Weight of 35S:FD2 High Overexpressing Lines Under Field Conditions

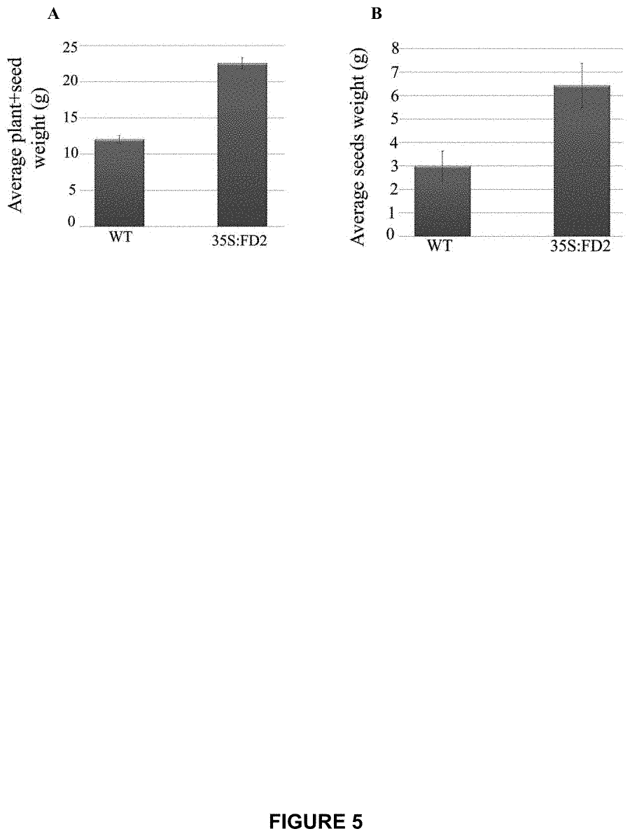

The present inventors performed gas exchange measurements under field conditions for a select 35S:FD2 high overexpressing line. As generally shown in FIG. 5, the yield from this exemplary 35S:FD2 line reveals a substantially greater biomass than the WT (100% increase), and shows substantially greater seed yield than the WT (100% increase).

### Example 6: Characterization of Excitation Energy Distribution Between PSII and PSI after Red Light Illumination in Wild-Type (A), and 35S:FD2 Overexpression Line

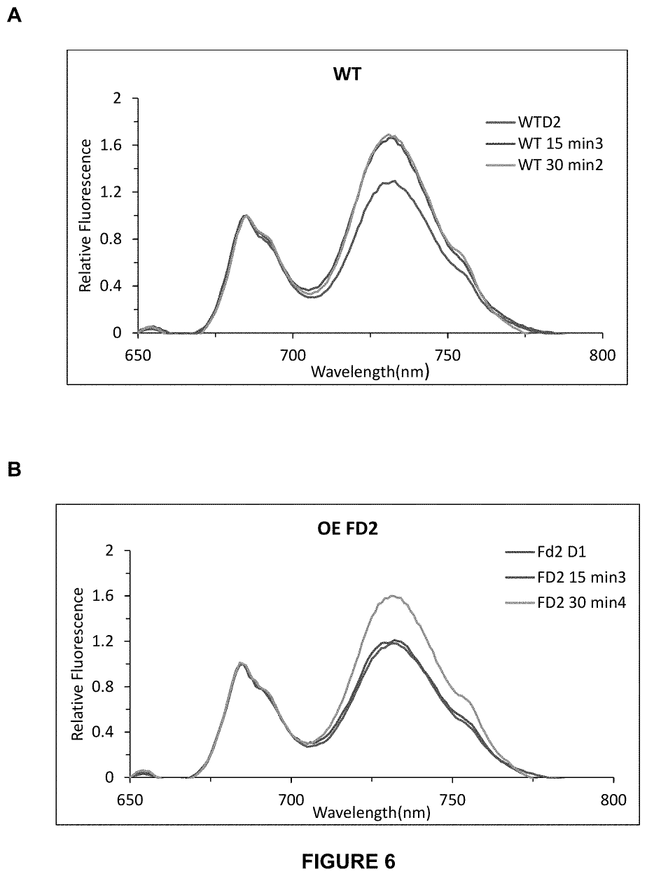

The present inventors characterized the between PSII and PSI after red light illumination in wild-type, and 35S:FD2 overexpression line. As demonstrated in FIGS. 6A-B, excitation energy distribution between photosystem complexes PSII and PSI, after red light illumination in wild-type (FIG. 6A), and 35S:FD2 overexpression line (FIG. 6B). As shown by the present inventors, dark adapted leaves after exposed to red light (100 μmol m⁻² s⁻¹) for 15 or 30 min were frozen in liquid nitrogen. Thylakoid isolation method was described by Mekala et al. (2015). Data represent mean values from 4 independent plants and error bars depict standard deviations. As shown in the figures, the present inventors demonstrated a delay in state II state transition indicating of more efficient linear electron transfer rates.

### Example 7: Characterization of the Effect of Chilling and/or High Light Stress on Maximal Photochemical Efficiency of PSII

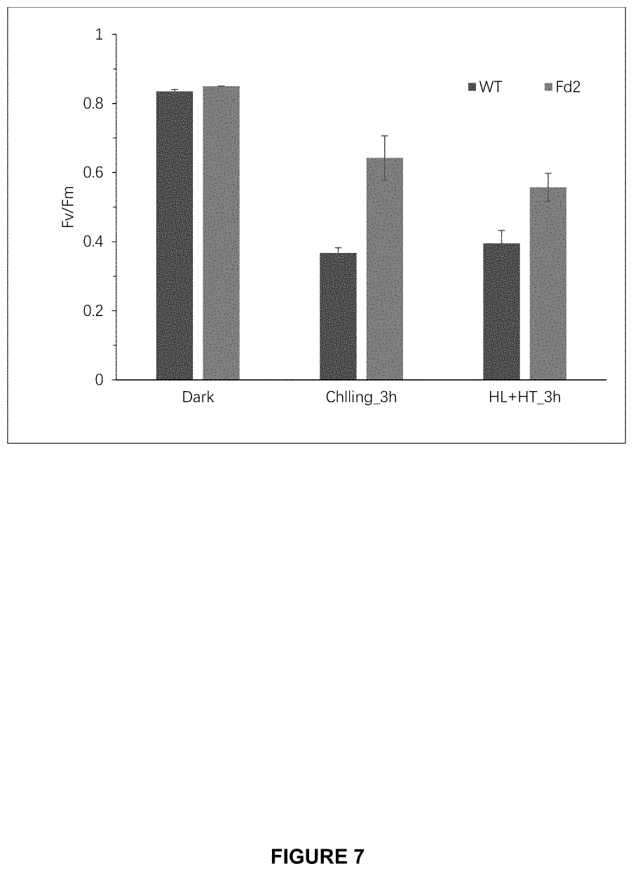

The present inventors characterized the effect of chilling or high light stress on maximal photochemical efficiency of PSII in leaves. As shown in FIG. 7, overexpressing Fd2 lines have as much as 50% reduction in loss of photosystem II (PSII) quantum efficiency following low or high temperature stress relative to WT. In this embodiment, dark adapted leaves were exposed to high light with high temperature (HL+HT: 2000 μmol m-2 s-1 at 37° C.) or chilling light (160 μmol m-2 s-1 at 7° C.) stresses for 3 h. Chlorophyll fluorescence was measured after 30 min dark recovery, by using Handy FluorCam FC 1000-H (Photon System Instruments). Data represent mean values from 4 independent plants and error bars depict standard deviations.

### Example 8: Characterization of the Effect of Chilling and/or High Light Stress on Non-Photochemical Quenching (NPQ)

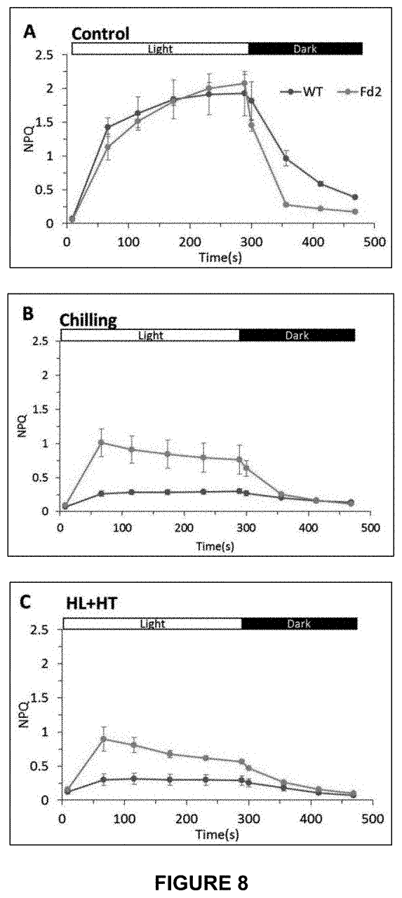

The present inventors characterized the effect of chilling and/or high light stress on non-photochemical quenching (NPQ) in leaves. As shown in FIG. 8, the present inventors demonstrated that the Fd2 overexpression lines have elevated NPQ and faster decay rates than WT consistent with improved photosynthetic efficiency and protection from temperature stress. FIGS. 8A, 8B and 8C shown NPQ development in control, chilling and high light (HL) with high temperature (HT) treated leaves, respectively. Data represent mean values from 4 independent plants and error bars depict standard deviations

### Example 9: Characterization of the Effect of Chilling and/or High Light Stress on Linear Electron Transport Rate (ETR)

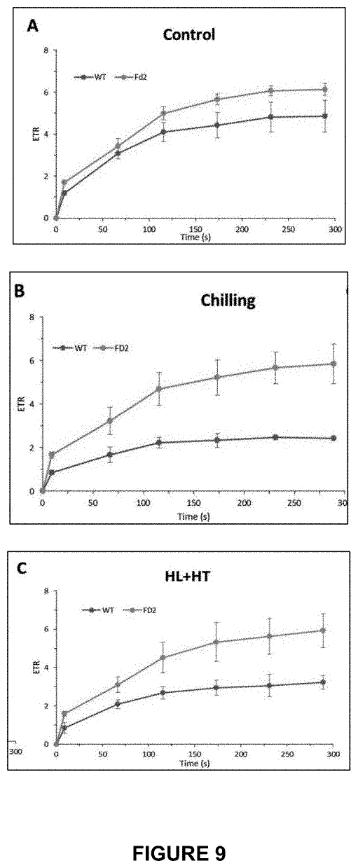

The present inventors characterized the effect and/of chilling or high light stress on linear electron transport rate (ETR) in leaves of Fd2 transgenic lines and WT. As shown in FIG. 9, the present inventors demonstrated that Fd2 transgenic lines have accelerated rates of ETR relative to WT and are more stress tolerant than WT. FIGS. 9A, 9B and 9C are NPQ development in control, chilling and high light (HL) with high temperature (HT) treated leaves, respectively. Data represent mean values from 4 independent plants and error bars depict standard deviations.

### Example 10: Contributions of Fd and FNR to Photosynthetic Electron Transport in (a) the Mesophyll Cell Chloroplasts and (b) the Bundle Sheath Cell Chloroplasts of Maize

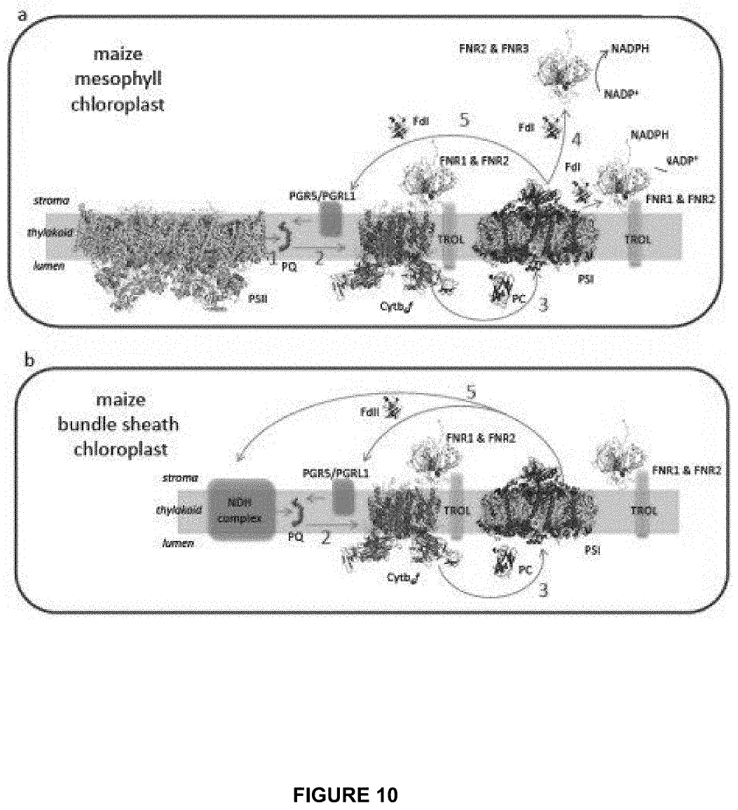

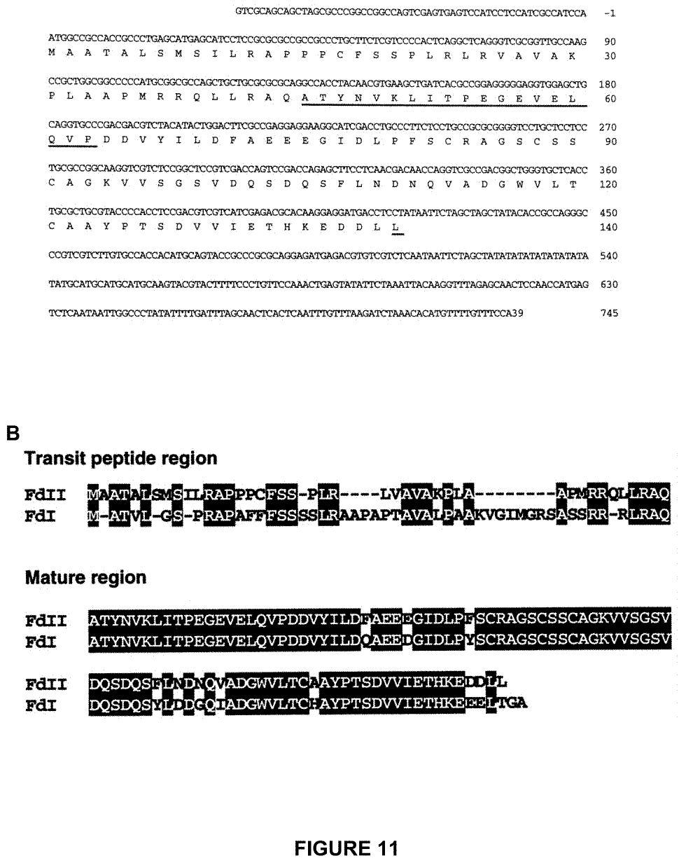

As generally shown in FIG. 10, In the linear photosynthetic electron transport chain, water is split at photosystem II (PSII), releasing electrons that are accepted by plastoquinone (PQ) (1), which transfers them to the cytochrome b₆f complex (Cytb₆f) (2). Plastocyanin (PC) carries these electrons through the thylakoid lumen to photosystem I (PSI) (3), where they are donated to ferredoxin (Fd). Fd can donate these electrons to multiple enzymes, including Fd:NADP(H) oxidoreductase (FNR) (4), which then photoreduces NADP⁺. In addition to this linear electron flow (LEF), Fd may return electrons to the membrane via either PGRL1 (the antimycin A sensitive pathway) or the NAD(P)H complex (NDH) dependent pathway (5). Both linear and cyclic electron flow generate the pH gradient necessary for ATP synthesis, but only the linear path results in release of electrons into stromal metabolism. Maize bundle sheath cells have very high rates of cyclic electron flow. This is facilitated by the presence of very little active PSII, a Fd iso-protein (Fd2) specific for the cyclic pathway, and elevated amounts of the NDH complex. Moreover, only two FNR iso-proteins, FNR1 and FNR2, are present, and these are tightly bound to the membrane by the thylakoid rhodanase like protein (TROL) and also associated with Cytb₆f, although their precise role in cyclic electron flow remains to be established. By contrast, the mesophyll cells have abundant, active PSII and relatively low amounts of the NDH complex. In combination with the specific Fd iso-protein, Fd1, this facilitates LEF. In the mesophyll three FNR proteins are present, FNR1, FNR2 and FNR3. All of FNR3 and a large proportion of FNR2 is soluble, and presumably involved in linear NADP⁺ photoreduction.

### Example 11: Immunoblot Analyses Ferredoxin (FD) Proteins Content in FD1 and/or FD2 Overexpression Lines

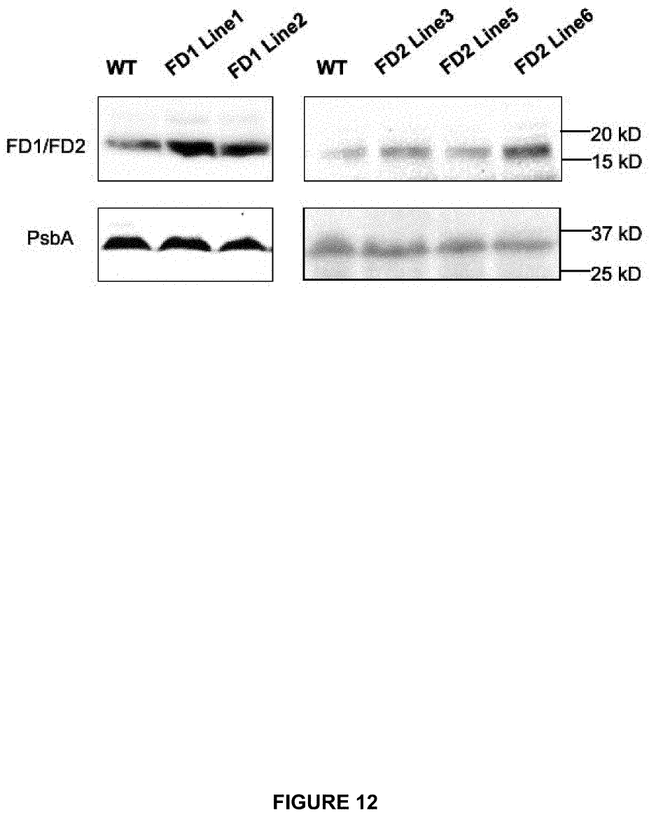

As generally shown in FIG. 12, the present inventors performed immunoblot analyses ferredoxin (FD) proteins content in FD1 and/or FD2 overexpression lines. FDx1 antibody was used against Camelina and Maize FD1 and/or maize FD2 proteins. Total protein was extracted using SDS sample buffer. The samples contained 4 μg total Chlorophyll and were separated by 4-20% precast polyacrylamide gel (Bio-Rad). PsbA and FD1/FD2 content was analyzed using anti-PsbA and anti-FDx1 antibodies from Agrisera. The present inventors demonstrate that FD proteins were enhanced in both in FD1 and FD2 overexpression lines relative to wild type. But no significant change was observed for the PsbA proteins (control).

### Example 12: P700 Oxidation and Reduction Kinetics in Maize FD1 Lines and Maize FD2 Lines

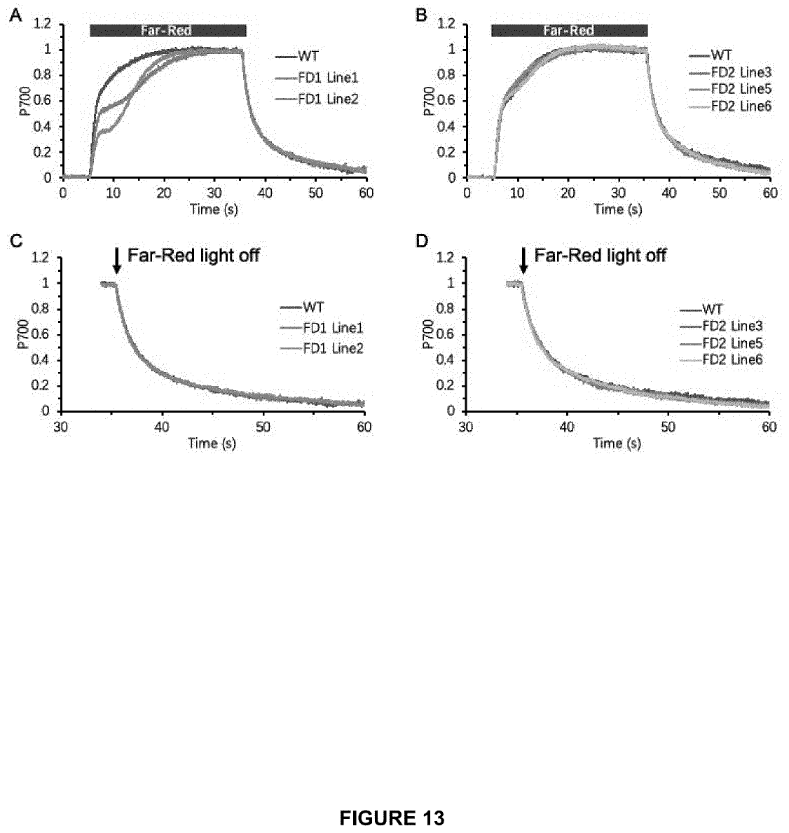

As generally shown in FIG. 13, the present inventors demonstrate P700 oxidation and reduction kinetics in (A) maize FD1, and (B) maize FD2 lines, respectively. The present inventors further provided a comparison of P700 reduction kinetics in maize FD1 lines (C) and maize FD2 lines (D). In this embodiment, plants were taken from the green house at 9:00 in morning and incubated in darkness for 3h. Data is presented as the average and standard deviation of three replicates. Overexpression of FD1 was associated with more rapid P700 oxidation during FR illumination compared to WT, but no difference was be found in P700 reduction consistent with accelerated linear election transfer rates. In FD2 lines, the present inventors did not observe any significant difference in P700 oxidation and reduction kinetics relative to wild type.

### Example 13: P700 Oxidation and Reduction Kinetics in DCMU Treated Maize FD1 and Maize FD2 Overexpression Lines

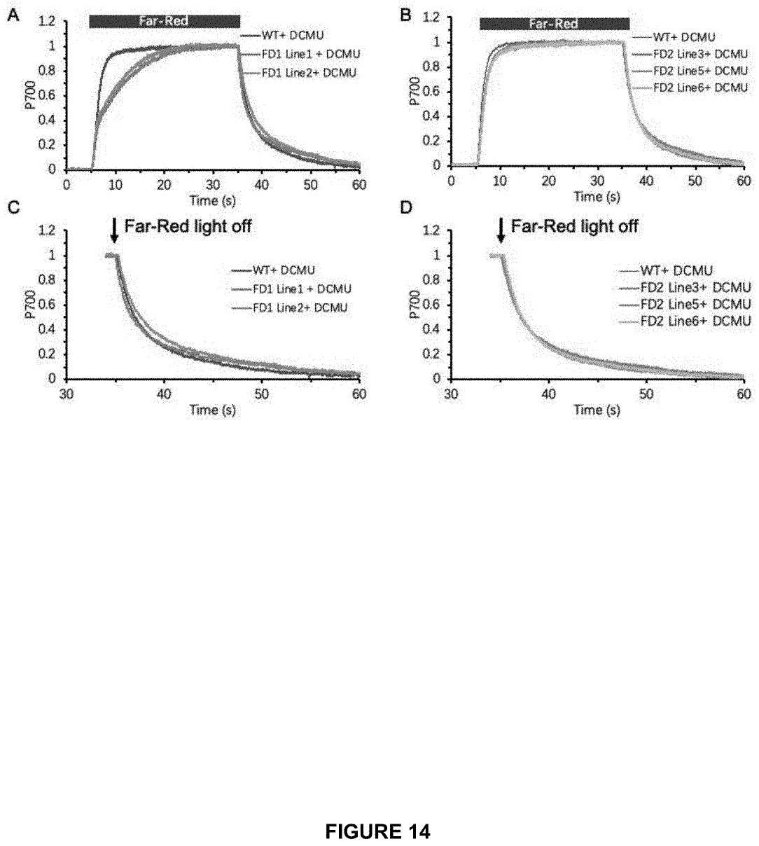

As generally shown in FIG. 14, the present inventors demonstrate P700 oxidation and reduction kinetics in DCMU treated maize FD1 (A) and maize FD2 (B) overexpression lines. Comparison of P700⁺ reduction kinetics in DCMU treated FD1 lines (C) and FD2 lines (D). Plants were taken from the green house at 9:00 in morning and incubated in darkness for 2 h followed by DCMU treatment for 1 h in darkness. Data is presented as the average and standard deviation of three replicates. After blocking linear electron transport (ETR) with DCMU, the present inventors observed results similar to FIG. 13, with the difference only observed being in P700 oxidation in FD1 overexpression lines compared to WT consistent with reductions in cyclic electron transfer. The present inventors further demonstrated that the FD2 lines had P700 oxidation and reduction kinetics similar to wild type.

### Example 14: Chlorophyll Fluorescence Fo Levels Increase During a Light to Dark Transition in FD1 and FD2 Overexpression Lines

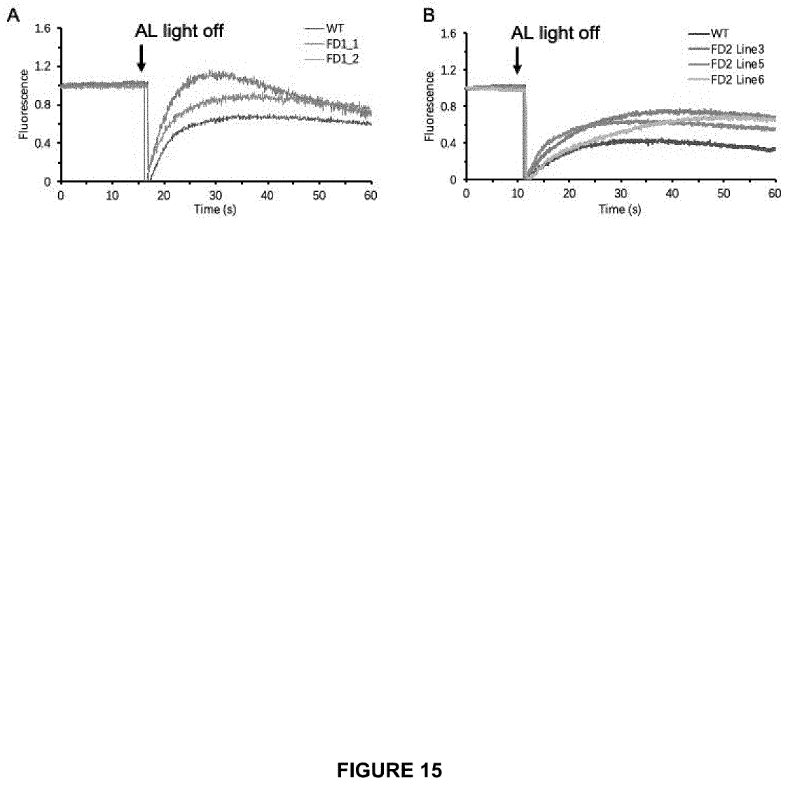

As generally shown in FIG. 15, the present inventors demonstrate chlorophyll fluorescence Fo levels increase during a light to dark transition in FD1 (A) and FD2 (B) overexpression lines. An increase in Fo chlorophyll fluorescence levels reflects electron donation from stromal reductants (ferredoxin) to the PQ pool. Exemplary model Camelina plants after 3 h dark adaptation were illuminated with actinic light (illumination intensity=220 μmol photons m⁻² s⁻¹) for 12 min, an increase in chlorophyll fluorescence Fo levels was observed during the dark post-illumination. As showed in FIG. 4A, an increase in Fo levels in the dark in FD1 lines was substantially greater and faster than for wild type. Similar results were observed for the FD2 lines shown in FIG. 15.

### Example 15: Alterations in Electron Transport Rates (ETR) in FD Overexpression Lines

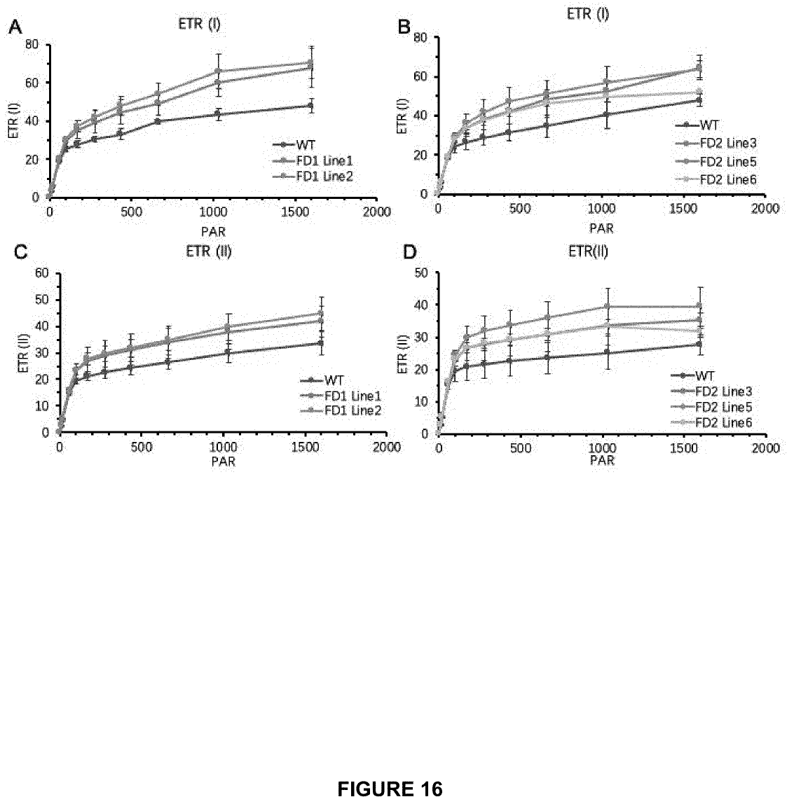

As generally shown in FIG. 16, the present inventors demonstrate alterations in electron transport rates (ETR) in FD overexpression lines. For example, graphs (A) and (B) are ETR around photosystem I (ETR (I)) in dark (3 h) treated FD1 and FD2 overexpression lines. Graphs (C) and (D) photosystem II (ETR (II)) in dark (3 h) treated FD1 and FD2 overexpression lines. In both FD1 and FD2 overexpression lines, the ETR(I) and ETR(II) are significantly higher than WT. These data demonstrate that overexpression of the FD protein was conducive to enhanced linear electron transport in the exemplary Camelina plants. Each data point represents the average of 3 values from independent plants, and error bars represent SD of three technical replicates.

### Example 16: Alterations in Non-Photochemical Quenching (NPQ) Induction in FD1 and FD2 Overexpression Lines

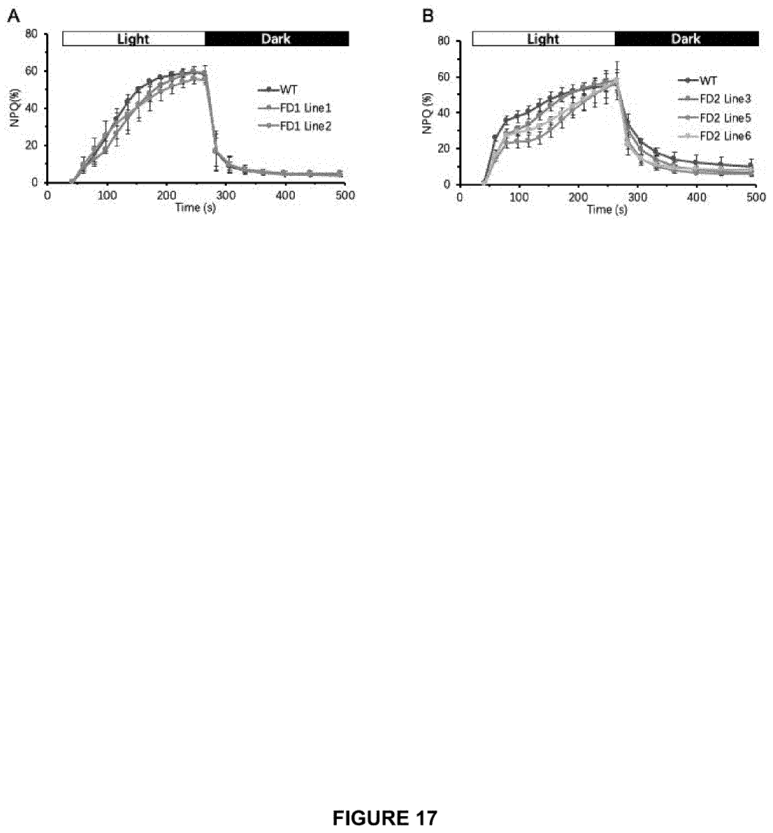

As generally shown in FIG. 17, the present inventors demonstrate alterations in non-photochemical quenching (NPQ) induction in FD1 (A) and FD2 (B) overexpression lines. Each data point represents the average 3 of values on independent plants, and error bars represent SD of three technical replicates. As shown, FD1 presented similar NPQ development and reduction kinetics relative to WT. In FD2 overexpressing lines, NPQ developed slower than WT, but had a faster reduction in the dark consistent with more efficient light utilization achieved through enhanced linear electron flow.

### Example 17: Gas Exchange Measurement of Greenhouse Grown Plants

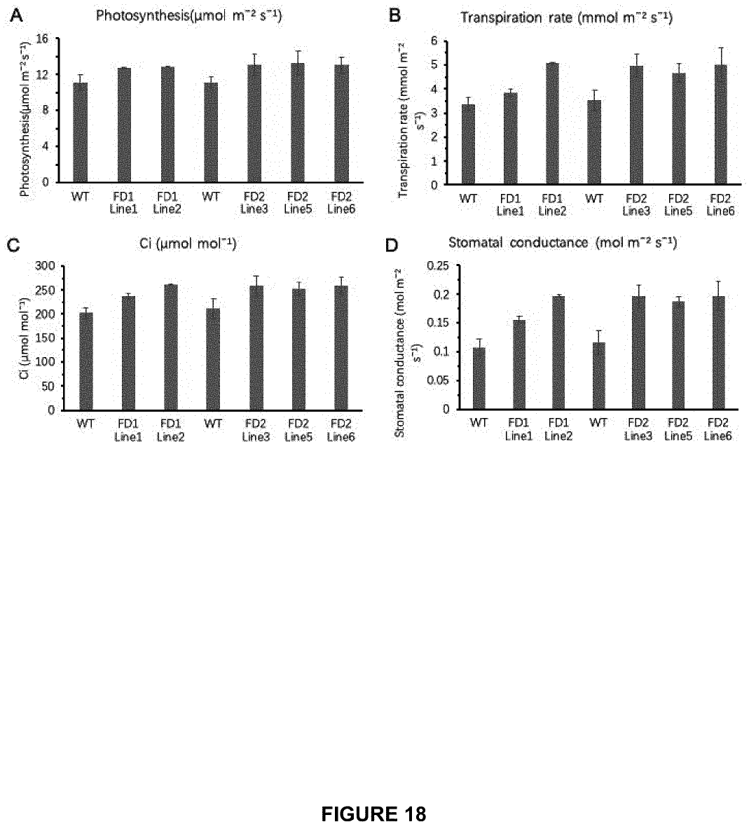

As generally shown in FIG. 18, the present inventors conducted gas exchange measurement of greenhouse grown plants. Notably, each data point represents the average of 3 to 6 values on independent plants, and error bars represent SD of 3 to 6 technical replicates. Two FD1 lines showed approximately a 14% increase in photosynthetic rates relative to WT (See FIG. 6A). The present inventors further demonstrated three FD2 overexpression lines that showed approximately 18% higher photosynthetic rate compared to WT. The transpiration rate in FD1 lines increased approximately 12% and 50% compared to WT. The three FD2 lines had approximately a 44% higher transpiration rate than WT. The measurements of internal leaf CO2 concentrations, or Ci in FD1 lines increased approximately 17% and 30% relative to WT demonstrating higher rates of photosynthesis. The three FD2 lines had approximately a 21% higher Ci than WT. The stomatal conductance in FD1 lines increased approximately 36% and 82% compared to WT. Finally, the three FD2 lines demonstrated approximately an 82% higher stomatal conductance than WT.

### Example 18: Field Trial Measurement for Photosynthetic CO₂ Gas Exchange Under Cloudy to Partially Sunny Weather Conditions

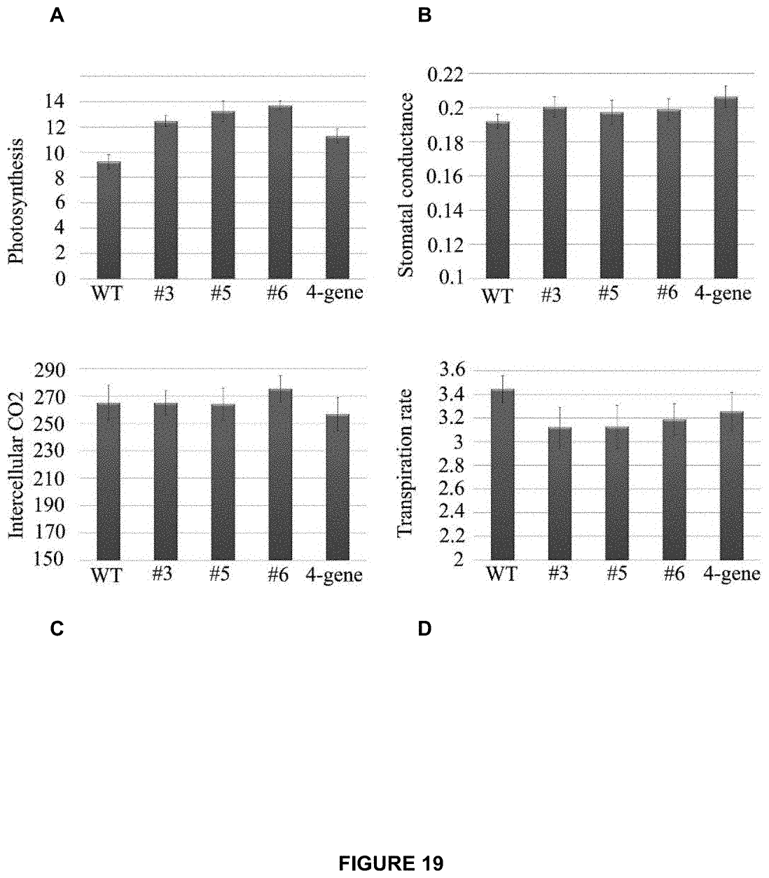

The present inventors conducted field trials in Santa Fe N. Mex. on a plurality of Fd2 transformants and 4-gene construct (HLA3, PGR5, LCIA, BCA) (See U.S. patent application Ser. No. 15/411,854). Photosynthetic CO₂ gas exchange measurements were taken in the field under cloudy to partially sunny weather conditions on the three best overexpressing performing Fd2 lines and one 4-gene construct line. As generally shown in FIG. 19, gas exchange measurements showed an increase of photosynthesis (approximately 30-35%) for the 35S:Fd2 transgenic lines and a 25% increase for the 4-gene construct relative to wild type. The present inventors also observed a 15% decrease of the transpiration rate for the 35S:Fd2 transgenic lines and a 10% decrease for the 4-gene construct.

### Example 19: Field Trial Measurement for Photosynthetic CO₂ Gas Exchange Under Sunny Weather Conditions

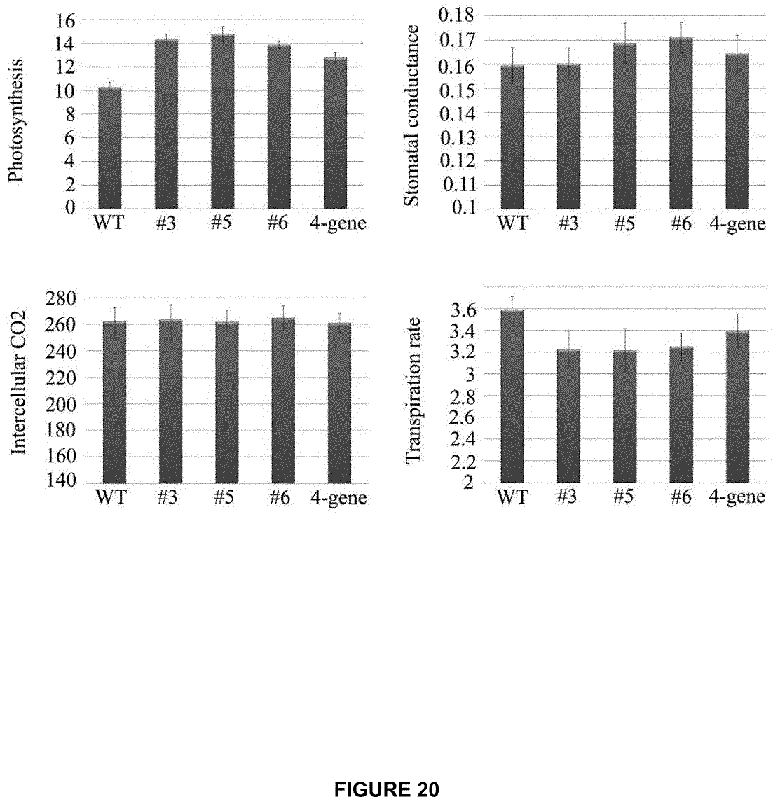

The present inventors conducted field trials in Santa Fe N. Mex. on a plurality of Fd2 transformants and 4-gene construct (HLA3, PGR5, LCIA, BCA). Photosynthetic CO₂ gas exchange measurements were taken in the field under cloudy to partially sunny weather conditions on the three best overexpressing performing Fd2 lines and one 4-gene construct line. As generally shown in FIG. 20, gas exchange measurements showed a 25% of increase for the 4-gene construct. The present inventors also observed a ˜10% decrease of the transpiration rate for the 4-gene construct.

### Example 20: First Harvest Biomass and Yield Production from Field Trials

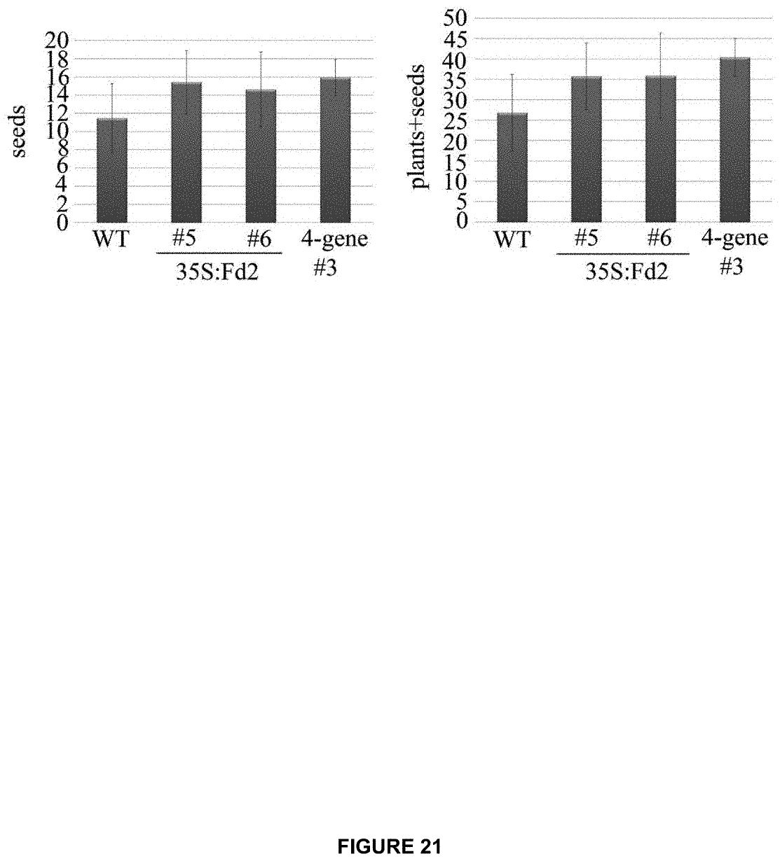

As generally demonstrated in FIG. 21, the present inventors conducted a first harvest of two overexpression ferredoxin (FD2) lines (#5, and #6) and one transgenic line (#3) of the 4-gene construct (PGR5/HLA3/BCA/LCIA). As shown in FIG. 21, (A) seed; and (B) plants+seeds measurements were performed and demonstrated a higher than WT seed and biomass production.

### Example 21: Second Harvest Biomass and Yield Production from Field Trials

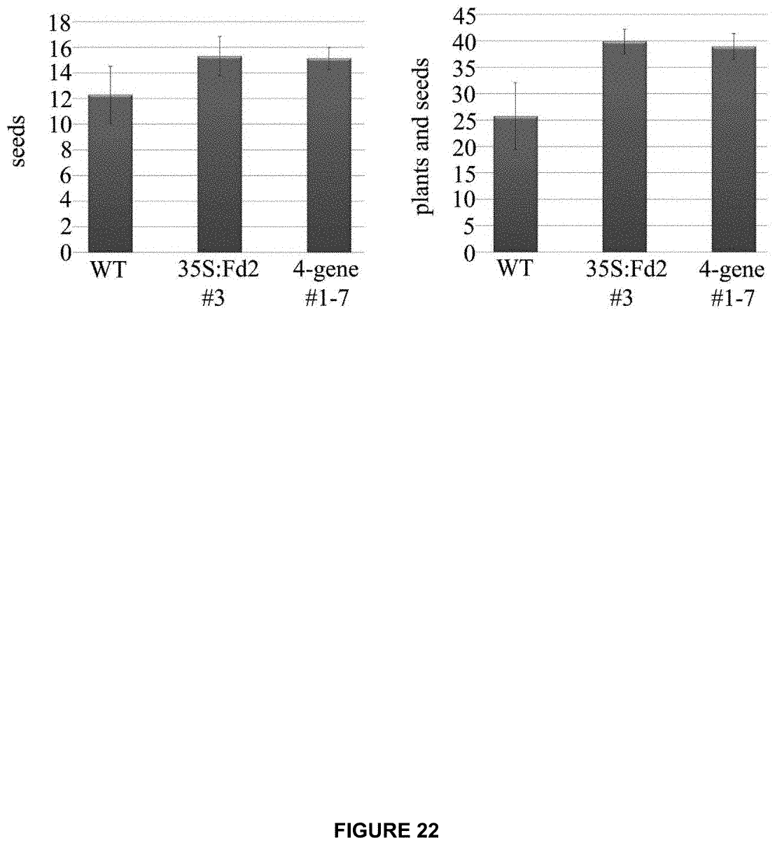

As generally demonstrated in FIG. 22, the present inventors conducted a second harvest of one overexpression ferredoxin line (#3) and one transgenic line (#1-7) of the 4-gene construct (PGR5/HLA3/BCA/LCIA). The Fd2 line had a 25% increase in seed yield and 60% increase in above ground biomass yield. As shown in FIG. 21, (A) seed; and (B) plants+seeds measurements were performed and demonstrated that the Fd2 line had a 25% increase in seed yield and 60% increase in above ground biomass yield.

### Example 22: Material and Methods

### Plants and Growth Condition

Wild-type (WT) Camelina sativa and ferredoxin1 (FD1) and 2 (FD2) T3 generation selfed plants were used in the experiments. Plants were grown in a greenhouse at 24° C./26° C. with a 14 h/10 h day/night photoperiod.

### SDS-PAGE and Immunoblot Analyses

Total protein was extracted by using SDS sample buffer (62.5 mM Tris-HCl (pH 6.8), 2.5% SDS, 0.7135M (5%) β-Mercaptoethanol and 10% glycerol) from dark 3 h adapted Camelina leaves. The chlorophyll concentration was determined in aqueous 80% acetone according to Porra (1989). The total protein equal to 4 μg total Chlorophyll were separated by 4-20% precast polyacrylamide gel (Bio-Rad). PsbA and FD1/FD2 content was analyzed by Anti-PsbA and Anti-FDx1 (Agrisera).

### Electron Transport Rate and NPQ Measurement

The parameters non-photochemical quenching (NPQ), electron transport rate around PSI (ETRI) and PSII (ETRII), and Fo rise were obtained using a Dual Pam 100 measuring system (Walz), from Camelina plants after 3 h dark adaptation.

### P700 Oxidation and Reduction Kinetics

P700 oxidation kinetics were determined by pre-illuminating the leaf with Far-red light (FR) and following P700+ reduction kinetics in the dark using Dual Pam 100 measuring system (Walz) with either 50 mM DCMU treated (60 min in darkness) or untreated leaves. Camelina plants were adapted in darkness for 3 h before P700 measurements.

### Gas Exchange Parameters

The photosynthetic CO₂ gas exchange rate, intercellular CO₂ concentration (Ci), stomatal conductance and transpiration (H₂O) rates in leaves were measured using the Li-Cor 6800 (Li-Cor Inc., United States) system in morning from 9:30 to 11:30 on days with optimal external conditions.

### REFERENCES

The following references are hereby incorporated in their entirety by reference:
[1] Arnon, D. I., Tagawa, K., and Tsujimoto, H. Y. (1963) Science 140, 378.
[2] Joliot, P., and Joliot, A. (2006) Biochim. Biophys. Acta 1757, 362-368.
[3] Shin, M., and Arnon, D. I. (1965) J. Biol. Chem. 240, 1405-1411.
[4] Knaff, D. B. (1996) in Advances in Photosynthesis. Oxygenic Photosynthesis: The Light Reactions (Ort, D. R., and Yocum, C. F., eds.) pp. 333-361, Kluwer Academic Publishers, Dordrecht.
[5] Montrichard, F., Alkhalfioui, F., Yano, H., Vensel, W. H., Hurkman, W. J., and Buchanan, B. B. (2009) J. Proteomics 72, 452-474.
[6] Suzuki, A., Oaks, A., Jacquot, J. P., Vidal, J., and Gadal, P. (1985) Plant Physiol. 78, 374-378.
[7] Hanke, G. T., and Hase, T. (2008) Photochem. Photobiol. 84, 1302-1309.
[8] Hase, T., Kimata, Y., Yonekura, K., Matsumura, T., and Sakakibara, H. (1991) Plant Physiol. 96, 77-83.
[9] Hanke, G. T., Kimata-Ariga, Y., Taniguchi, I., and Hase, T. (2004) Plant Physiol. 134, 255-264.
[10] Onda, Y., Matsumura, T., Kimata-Ariga, Y., Sakakibara, H., Sugiyama, T., and Hase, T. (2000) Plant Physiol. 123, 1037-1045.
[11] Kimata-Ariga, Y., Matsumura, T., Kada, S., Fujimoto, H., Fujita, Y., Endo, T., Mano, J., Sato, F., and Hase, T. (2000) EMBO J. 19, 5041-5050.
[12] Kimata, Y., and Hase, T. (1989) Plant Physiol. 89, 1193-1197.
[13] Matsumura, T., Kimata-Ariga, Y., Sakakibara, H., Sugiyama, T., Murata, H., Takao, T., Shimonishi, Y., and Hase, T. (1999) Plant Physiol. 119, 481-488.
[14] Voss, I., Koelmann, M., Wojtera, J., Holtgrefe, S., Kitzmann, C., Backhausen, J. E., and Scheibe, R. (2008) Physiol. Plant 133, 584-598.
[15] Lehtimäki N1, Lintala M, Allahverdiyeva Y, Aro E M, Mulo P., (2010) J Plant Physiol. 167, 1018-22.
[16] Blanco N E, Ceccoli R D, Vía M V D, et al. Expression of the Minor Isoform Pea Ferredoxin in Tobacco Alters Photosynthetic Electron Partitioning and Enhances Cyclic Electron Flow. Plant Physiology. 2013; 161(2): 866-879.
[17] Tomohiro M., Yoko K.-A., Hitoshi S., Tatsuo S.a, Hiroshi M., Toshifumi T., Yasutsugu S.i, Toshiharu H., Plant Physiology February 1999, 119 (2) 481-488.
[18] Voss, I., Koelmann, M., Wojtera, J Holtgrefe, S., Kitzmann, C., Backhausen, J. E. and Scheibe, R. (2008), Knockout of major leaf ferredoxin reveals new redox-regulatory adaptations in Arabidopsis thaliana. Physiologia Plantarum, 133: 584-598.
[19] Voss I, Goss T, Murozuka E, et al. FdC1, a Novel Ferredoxin Protein Capable of Alternative Electron Partitioning, Increases in Conditions of Acceptor Limitation at Photosystem I. The Journal of Biological Chemistry. 2011; 286(1):50-59.
[20] Kimata-Ariga Y, Matsumura T, Kada S, et al. Differential electron flow around photosystem I by two C₄-photosynthetic-cell-specific ferredoxins. The EMBO Journal. 2000; 19(19):5041-5050.
[21] Yamamoto H, Peng L, Fukao Y, Shikanai T. An Src Homology 3 Domain-Like Fold Protein Forms a Ferredoxin Binding Site for the Chloroplast NADH Dehydrogenase-Like Complex in Arabidopsis. The Plant Cell. 2011; 23(4): 1480-1493.
[22] Hanke G T, Kimata-Ariga Y, Taniguchi I, Hase T. A Post Genomic Characterization of Arabidopsis Ferredoxins. Plant Physiology. 2004; 134(1): 255-264.
[23] Curr Protein Pept Sci. 2014 June; 15(4): 385-393.

### SEQUENCE LISTINGS

As noted above, the instant application contains a full Sequence Listing which has been submitted electronically in ASCII format and is hereby incorporated by reference in its entirety. The following sequences are further provided herewith and are hereby incorporated into the specification in their entirety:

Amino Acid
Ferredoxin 2 (Fd2)
Zea Mays
SEQ ID NO. 1
MAATALSMSILRAPPPCFSSPLRLRVAVAKPLAAPMRRQLLRAQ
ATYNVKLITPEGEVELQVPDDVYILDFAEEEGIDLPFSCRAGSC
SSCAGKVVSGSVDQSDQSFLNDNQVADGWVLTCAAYPTSDVVIE
THKEDDLL
Amino Acid
Ferredoxin 1 (Fd1)
Zea Mays
SEQ ID NO. 2
MATVLGSPRAPAFFFSSSSLRAAPAPTAVALPAAKVGIMGRSAS
SRRRLRAQATYNVKLITPEGEVELQVPDDVYILDQAEEDGIDLP
YSCRAGSCSSCAGKVVSGSVDQSDQSYLDDGQIADGWVLTCHAY
PTSDVVIETHKEEELTGA
Amino Acid
Ferredoxin 1 (Fd1) varient1
Zea Mays
SEQ ID NO. 3
MATVLGSPRAPAFFFSPSSLRAAPAPTAVALPAAKVGIMGRSAS
SRGRLRAQATYNVKLITPEGEVELQVPDDVYILDQAEEDGIDLP
YSCRAGSCSSCAGKVVSGSVDQSDQSYLDDGQIAAGWVLTCHAY
PTSDVVIETHKEEELTGA
DNA
Ferredoxin 2 (Fd2) mRNA
Zea Mays
SEQ ID NO. 4
ATGGCCGCCACCGCCCTGAGCATGAGCATCCTCCGCGCGCCGCC
GCCCTGCTTCTCGTCCCCACTCAGGCTCAGGGTCGCGGTTGCCA
AGCCGCTGGCGGCCCCCATGCGGCGCCAGCTGCTGCGCGCGCAG
GCCACCTACAACGTGAAGCTGATCACGCCGGAGGGGGAGGTGGA
GCTGCAGGTGCCCGACGACGTCTACATACTGGACTTCGCCGAGG
AGGAAGGCATCGACCTGCCCTTCTCCTGCCGCGCGGGGTCCTGC
TCCTCCTGCGCCGGCAAGGTCGTCTCCGGCTCCGTCGACCAGTC
CGACCAGAGCTTCCTCAACGACAACCAGGTCGCCGACGGCTGGG
TGCTCACCTGCGCTGCGTACCCCACCTCCGACGTCGTCATCGAG
ACGCACAAGGAGGATGACCTCCTATAA
DNA
Ferredoxin 2 (Fd2) full gene
Zea Mays
SEQ ID NO. 5
GTGTGGCCGCCCGTGTCGTGTAGTGTGTAGTCGCAGCAGCTAGC
GCCCGGCCGGCCAGTCGAGTGAGTCCATCCTCCATCGCCATCCA
ATGGCCGCCACCGCCCTGAGCATGAGCATCCTCCGCGCGCCGCC
GCCCTGCTTCTCGTCCCCACTCAGGCTCAGGGTCGCGGTTGCCA
AGCCGCTGGCGGCCCCCATGCGGCGCCAGCTGCTGCGCGCGCAG
GCCACCTACAACGTGAAGCTGATCACGCCGGAGGGGGAGGTGGA
GCTGCAGGTGCCCGACGACGTCTACATCCTGGACTTCGCCGAGG
AGGAAGGCATCGACCTGCCCTTCTCCTGCCGTGCGGGGTCCTGC
TCCTCCTGCGCCGGCAAGGTCGTCTCTGGCTCCGTCGACCAGTC
CGACCAGAGCTTCCTCAACGACAACCAGGTCGCCGACGGTTGGG
TGCTCACCTGCGCTGCGTACCCCACCTCCGACGTCGTCATCGAG
ACGCACAAGGAGGATGACCTCCTATAATTCTAGCTAGCTATACA
CCGCCAGGGCCCGTCGTCTTGTGCCACCACATGCAGTACCGCCC
GCGCAGGAGATGAGACGTGTCGTCTCAATAATTCTAGCTATATA
TATATATATGCATGCATGCATGTACTTTTCCCTGTTCCAAACTG
AGTATATTCTAAATTACAAGATTTAATCACAAGGTTTAGAGCAA
CTCCAACCATGAGTCTCATAATTGGCTCTATATTTTGATTTAGC
AACTCACTTAATTTGTTTAAGATCTAAACACATGTTTTGTTTC
DNA
Ferredoxin 1 (Fd1) mRNA
Zea Mays
SEQ ID NO. 6
ATGGCCACCGTCCTGGGCAGCCCCCGCGCGCCGGCCTTCTTCTT
CTCGTCGTCCTCCCTCCGCGCCGCGCCGGCGCCTACCGCCGTGG
CGCTGCCTGCGGCCAAGGTGGGCATCATGGGCCGTAGCGCCAGC
AGCAGGCGCAGGCTGCGCGCGCAGGCCACCTACAACGTGAAGCT
GATCACGCCAGAGGGGGAGGTGGAGCTGCAGGTGCCCGACGACG
TGTACATCCTGGACCAGGCCGAGGAGGACGGCATCGACCTGCCC
TACTCCTGCCGCGCGGGGTCCTGCTCCTCGTGCGCCGGCAAGGT
CGTCTCCGGCTCCGTGGACCAGTCCGACCAGAGCTACCTCGACG
ACGGCCAGATCGCCGACGGCTGGGTGCTCACCTGCCACGCCTAC
CCCACCTCTGACGTCGTCATCGAGACGCACAAGGAGGAGGAGCT
CACCGGCGCATAA
DNA
Ferredoxin 1 (Fd1) mRNA varient1
Zea Mays
SEQ ID NO. 7
ATGGCCACCGTCCTAGGCAGCCCCCGCGCGCCGGCCTTCTTCTT
CTCGCCGTCCTCCCTCCGTGCCGCGCCGGCGCCCACCGCCGTGG
CGCTGCCTGCGGCCAAGGTGGGCATCATGGGCCGTAGCGCCAGC
AGCAGGGGCAGGCTGCGCGCGCAGGCCACCTACAACGTGAAGCT
GATCACGCCGGAGGGGGAGGTGGAGCTGCAGGTGCCCGACGACG
CTGTACATCCTGGACCAGGCCGAGGAGGACGGCATCGACCTGCC
CTACTCCTGCCGCGCGGGGTCCTGCTCCTCCTGCGCCGGCAAGG
TCGTCTCCGGCTCCGTGGACCAGTCCGACCAGAGCTACCTCGAG
ACGGCCAGATCGCCGCCGGCTGGGTGCTCACCTGCCACGCCTAC
CCCACCTCTGACGTCGTCATCGAGACGCACAAGGAGGAGGAGCT
CACCGGCGCATAA
Amino Acid
Photosynthetic ferredoxin Variant1
Sorghum bicolor
SEQ ID NO. 8
MATALSSLRAPAAFSLGIAAAPAPAAAATVVALPAAKPARGARL
RAQATYNVKLITPDGEVELQVPDDVYILDQAEEEGIDLPFSCRA
GSCSSCAGKVVSGTVDQSDQSFLDDAQVEGGWVLTCAAYPTSDV
VIETHKEEDLVG
Amino Acid
Photosynthetic ferredoxin Varient2
Saccharum hybrid
SEQ ID NO. 9
MSTSTFATSCTLLGNVRTQASQAAVKSPSSLSFFSQVMKVPSLK
TSKKLDVSAMAVYKVKLVTPEGQEHEFDAPDDTYILDAAETAGV
ELPYSCRAGACSTCAGKIESGAVDQSDGSFLDDGQQEEGYVLTC
VSYPKSDCAIHTHKEGDLY
Amino Acid
Photosynthetic ferredoxin Varient2
Panicum hallii
SEQ ID NO. 10
MSISTFATSCVLLSNVRTQTSQTPVKSPSSLSFFSQGMKVPSLK
TSKKLDVSAMAVYKVKLVTPEGVEHEFEAPDDTYILDAAETAGV
ELPYSCRAGACSTCAGKIEAGEVDQSDGSFLDDGQQAEGYVLTC
VSYPKSDCVIHTHKEGDLY
Amino Acid
Ferredoxin 2 (Fd2)
Arabidopsis thaliana
SEQ ID NO. 11
MASTALSSAIVGTSFIRRSPAPISLRSLPSANTQSLFGLKSGTA
RGGRVTAMATYKVKFITPEGELEVECDDDVYVLDAAEEAGIDLP
YSCRAGSCSSCAGKVVSGSVDQSDQSFLDDEQIGEGFVLTCAAY
PTSDVTIETHKEEDIV
DNA
Ferredoxin 2 (Fd2)
Arabidopsis thaliana
SEQ ID NO. 12
GTGTGAGCTGTCCCAAGTAAGACCACGTAATACTCACCTCAACA
AGATAGTGTTCTTAAAGTGTGTCAAACACAATCACACACACACA
AATCATAAAACACAAAGACGATAATCCATCGATCCACAGAATAG
ACGCCACGTGGTAGATAGGATTCTCACTAAAAAGTTCTCACCTT
TTAATCTTTCTCCACGCCATTTCCACAAGCCATAATCCTCAAAA
ATCTCAACTTTATCTCCCAAAACACAAAACAAAAAAAAATGGCT
TCCACTGCTCTCTCAAGCGCCATCGTCGGAACTTCATTCATCCG
CTCGTTCCCCAGCTCCAATCAGTCTCCGTTCCCTTCCATCAGCA
ACACACAATCCCTCTTCGGTCTCAAATCAGGCACCGCTCGTGGT
GGACGTGTCACAGCCATGGCTACATACAAGGTCAAGTTCATCAC
ACCAGAAGGTGAGCTAGAGGTTGAGTGTGACGACGACGTCTACG
TTCTTGATGCTGCTGAGGAAGCTGGAATCGATTTGCCTTACTCT
TTGCCGTGCTGGTTCTTGTTCGAGCTGTGCTGGAAAGTTGTGTC
TGGATCTGTTGATCAGTCTGACCAGAGTTTCCTTGATGATGAAC
AGATTGGTGAAGGGTTTGTTCTCACTTGTGCTGCTTACCCTACC
TCTGATGTTACCATTGAAACCCACAAAGAAGAAGACATTGTTTA
AGCCTCACCTACTCACCAGCTTTTGATGGTTTAAAAATCATGTC
TTTATAAATTTCACATTTTGGGTTGAGTTTGTTGTTACTAAAAA
CTATTGTTATCTGTTGTTATTGTTCCTGGTTTGGCTCACCATCA
ATCGATGACATTTTAAACTATGCAACTGCAAATTCTGCAACACT
TTCGATGAGAATCTAACATTATCGTTTAAACATTGGAAATACAT
TTTCTTGAAGTCTAGCTAGCTTTGGTTTGTAGTTCTTATTCTGA
ACTCAACAATCATCAAA

1. A transgenic C3 plant expressing a heterologous polynucleotide sequence operably linked to a promoter sequence encoding at least one of the following:
photosynthetic ferredoxin-1 (Fd1) protein that enhances linear electron transport (LET) in said transgenic C3 plant;
photosynthetic ferredoxin-2 (Fd2) protein that enhances photosynthetic linear electron transport (LET) in said transgenic C3 plant; and
a combination of said photosynthetic Fd1 and Fd2 proteins.

2. The transgenic plant of claim 1 wherein said photosynthetic Fd1 protein is from a C4 plant and further comprises a mesophyll cell specific photosynthetic Fd1 protein from a C4 plant.

3. The transgenic plant of claim 2 wherein said photosynthetic Fd1 protein from a C4 plant is selected from the group consisting of: SEQ ID NO. 2, SEQ ID NO. 3, and an Fd1 variant thereof.

4. The transgenic plant of claim 2 wherein said heterologous nucleotide sequence encoding a C4 photosynthetic Fd1 protein is selected from the group consisting of: SEQ ID NO 6, SEQ ID NO. 7, and a nucleotide sequence having 85% sequence identity with at least one of said nucleotide sequences.

5. The transgenic plant of claim 3 wherein said photosynthetic Fd1 protein enhances photosynthetic cyclic electron transport (CET) in said transgenic plant.

6. The transgenic plant of claim 1 wherein said photosynthetic Fd2 protein is from a C4 plant and further comprises a bundle sheath cell specific Fd2 protein from a C4 plant.

7. The transgenic plant of claim 6 wherein said photosynthetic Fd2 protein from a C4 plant is selected from the group consisting of: SEQ ID NO. 1, or an Fd2 variant thereof.

8. The transgenic plant of claim 6 wherein said heterologous nucleotide sequence encoding a C4 photosynthetic Fd2 protein is selected from the group consisting of: SEQ ID NO 4, SEQ ID NO. 5, and a nucleotide sequence having 85% sequence identity with at least one of said nucleotide sequences.

9. The transgenic plant of claim 7 wherein said photosynthetic Fd2 protein enhances photosynthetic linear electron transport (LET) in said transgenic plant.

10. The transgenic plant of claim 1 wherein said transgenic plant is selected from the group consisting of: a C3 oil seed crop, a C3 oil crop, and a C3 food crop, Camelina sativa, Cannabis, hemp.

11-12. (canceled)

13. The transgenic plant of claim 1 wherein said transgenic plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.

14. (canceled)

15. The transgenic plant of claim 13 wherein said photosynthetic Fd protein is a photosynthetic Fd protein from a C4 plant.

16. The transgenic plant of claim 15 wherein the C4 plant is selected from the group consisting of selected from the group consisting of: a C4 plant from the genera Panicum, a C4 plant from the genera Saccharum, a C4 plant from the genera Setaria, a C4 plant from the genera sorghum and a C4 plant from the genera Zea.

17. The transgenic plant of claim 15 wherein said photosynthetic Fd protein from a C4 plant is selected from the group consisting of: a bundle sheath cell specific photosynthetic Fd protein from a C4 plant, and mesophyll cell specific photosynthetic Fd protein from a C4 plant.

18. The transgenic plant of claim 17 wherein said photosynthetic Fd protein from a C4 plant is selected from the group consisting of: photosynthetic ferredoxin-1 (Fd1) protein from a C4 plant, and photosynthetic ferredoxin-2 (Fd2) protein from a C4 plant.

19. The transgenic plant of claim 18 wherein said photosynthetic Fd1 protein is from maize (Zea mays).

20. The transgenic plant of claim 18 wherein said photosynthetic Fd2 protein is from maize (Zea mays).

21. The transgenic plant of claim 18 wherein said photosynthetic Fd2 protein enhances linear electron transfer rates in said transgenic plant.

22. The transgenic plant of claim 18 wherein said photosynthetic Fd1 protein enhances photosynthetic cyclic electron transport (CET) in said transgenic plant.

23. The transgenic plant of claim 18 wherein said Fd1 protein has an amino acid sequence with at least 85% identity to an amino acid sequence selected from the group consisting of: SEQ ID NO. 2, and SEQ ID NO. 3.

24. The transgenic plant of claim 18 wherein said Fd2 protein has an amino acid sequence with at least 85% identity to an amino acid sequence according to SEQ ID NO. 1.

25. The transgenic plant of claim 1 wherein said heterologous polynucleotide sequence encoding a photosynthetic Fd protein comprises a heterologous polynucleotide sequence encoding photosynthetic Fd protein from a C4 plant.

26. The transgenic plant of claim 25 wherein said a heterologous polynucleotide sequence encoding photosynthetic Fd protein from a C4 plant comprises a nucleic acid sequence selected from the group consisting of: SEQ ID NOs. 4, 5, 6, 7, and a nucleotide sequence having 85% sequence identity with at least one of said nucleotide sequences.

27. The transgenic plant of claim 15 wherein said photosynthetic Fd protein has an amino acid sequence with at least 85% identity to an amino acid sequence selected from the group consisting of: SEQ ID NO. 8, SEQ ID NO. 9, and SEQ ID NO. 10.

28-29. (canceled)

30. The transgenic plant of claim 13 wherein the C3 transgenic plant is selected from the group consisting of: a C3 oilseed crop, a C3 oil crop, and a C3 food crop, Camelina sativa, Cannabis, hemp.

31-92. (canceled)

92. A method of enhancing photosynthesis comprising the step of transforming a C3 plant by introducing an expression cassette comprising a heterologous polynucleotide sequence operably linked to a promoter sequence encoding at least one of the following:
photosynthetic ferredoxin-1 (Fd1) protein from a C4 plant that enhances linear electron transport (LET) in said transgenic plant; and
photosynthetic ferredoxin-2 (Fd2) protein from a C4 plant that enhances photosynthetic linear electron transport (LET) in said transgenic plant.

93. The method of claim 92 wherein said photosynthetic Fd2 protein comprises a photosynthetic Fd2 protein selected from the group consisting of: an amino acid sequence according to SEQ ID NO. 1, and an Fd2 variant thereof.

94. The method of claim 92 wherein said photosynthetic Fd1 protein comprises a photosynthetic Fd1 protein selected from the group consisting of: an amino acid sequence according to SEQ ID NO. 2, an amino acid sequence according to SEQ ID NO. 3, and an Fd1 variant thereof.

95. The method of claim 92 wherein said photosynthetic Fd1 sequence comprises a polynucleotide sequence selected from the group consisting of: SEQ ID NO. 6, SEQ ID NO. 7, or a polynucleotide having at least 85% sequence identity to SEQ ID NO. 6, or SEQ ID NO. 7.

96. The method of claim 94 wherein said transformed C3 plant is selected from the group consisting of: a C3 oil seed crop, a C3 oil crop, and a C3 food crop.

97. The method of claim 94 wherein said transformed C3 plant is selected from the group consisting of: Cannabis, and hemp.

98. The method of claim 92 wherein said transformed C3 plant exhibits at least one of the following phenotypes compared to a control plant:
enhanced photosynthetic efficiency;
enhanced photosynthetic electron transfer rates;
enhanced photosynthetic CO₂ fixation;
enhanced abiotic stress tolerance;
enhanced plant yield; and
enhanced plant biomass.

99-147. (canceled)
